(19) United States
(12) Patent Application Publication
(10) Pub. No.: US 2019/0329063 A1
(43) Pub. Date: Oct. 31, 2019

(54) METHOD FOR MODIFYING THE THERAPEUTIC EFFECTS OF DRUGS

(71) Applicant: The Medical Research, Infrastructure and Health Services Fund of the Tel Aviv Medical Center, Tel-Aviv (IL)

(72) Inventors: Talma HENDLER, Tel-Aviv (IL); Gal RAZ, Kfar Daniel (IL); Roy Itzhak SAR-EL, Tel-Aviv (IL)

(73) Assignee: The Medical Research, Infrastructure and Health Services Fund of the Tel Aviv Medical Center, Tel-Aviv (IL)

(21) Appl. No.: 16/473,256

(22) PCT Filed: Dec. 21, 2017
(86) PCT No.: PCT/IB17/58300

§ 371 Date: Jun. 25, 2019

Related U.S. Application Data
(60) Provisional application No. 62/439,041, filed on Dec. 25, 2016.

(51) Int. Cl. A61N 2/00; A61K 31/4458; A61K 31/198; A61K 31/4515; A61G 10/02; A61N 1/36; A61M 21/02

(52) U.S. Cl. CPC A61N 2/002; A61K 31/4458; A61K 31/198; A61M 2021/0044; A61G 10/026; A61N 1/36025; A61M 21/02; A61K 31/4515

(57) ABSTRACT

A method for modifying the effect of a drug by application of an activation protocol, comprising:
administering a drug according to a treatment protocol;
applying an activation protocol in a timed relationship to the administering, for differentially activation of at least one selected brain region;
wherein the differentially activation allows the drug to selectively interact with the at least one selected brain region.

### RELATED APPLICATION

This application claims the benefit of priority under 35 USC § 119(e) of U.S. Provisional Patent Application No. 62/439,041 filed 25 Dec. 2016, the contents of which are incorporated herein by reference in their entirety.

### FIELD AND BACKGROUND OF THE INVENTION

The present invention, in some embodiments thereof, relates to a method for modifying the effect of a drug and, more particularly, but not exclusively, to a method for modifying the effect of psychotropic drugs.

### SUMMARY OF THE INVENTION

Following are some examples of some embodiments of the invention:
Example 1. A method for modifying the effect of a drug by application of an activation protocol, comprising:

administering a drug according to a treatment protocol;

applying an activation protocol in a timed relationship to said administering, for selectively activation of at least one selected brain region;

wherein said selectively activation allows said drug to selectively interact with said at least one selected brain region.
Example 2. The method of example 1, wherein said applying further comprises applying an activation protocol before said administering.
Example 3. The method of example 1, wherein said applying further comprises applying an activation protocol after said administering.
Example 4. The method of any of the previous examples, further comprising determining a brain activation profile prior to said applying.
Example 5. The method of any of the previous examples, further comprising determining a brain activation profile following said applying.
Example 6. The method of examples 4 or 5, further comprising modifying said activation protocol according to said determining.
Example 7. The method of any one of examples 4 to 6, further comprising modifying said drug dosage according to said determining.
Example 8. The method of any of the previous examples, further comprising determining the effect of said drug by measuring at least one clinical parameter value, following said applying.
Example 9. The method of example 8, wherein said clinical parameter is selected from a group comprising skin conductance, heart rate, blood pressure or blood flow, pupil diameter.
Example 10. The method of examples 8 or 9, further comprising modifying said activation protocol if said effect of said drug is not a desired effect.
Example 11. The method of example 1, wherein said timed relationship is adjusted according to said drug.
Example 12. The method of example 5, wherein said timed relationship is adjusted according to said effect.
Example 13. The method of example 1, wherein said timed relationship is at least 5 minutes before or after said applying.
Example 14. The method of example 8, wherein said effect comprises reducing at least one side effect of said drug.
Example 15. The method of any of the previous examples, wherein said drug is methylphenidate and wherein said treatment protocol is used to treat ADHD.
Example 16. The method of example 15, wherein said methylphenidate dosage is in the range of 0.5-30 mg.
Example 17. The method of examples 15 or 16, wherein said activation protocol increases the activation level of at least one brain region selected from the list of: right/left dorsolateral prefrontal cortex ventrolateral prefrontal cortex, parietal lobule.
Example 18. The method of any one of examples 15 to 17, wherein said activation protocol decreases the activation level of at least one brain region, selected from the list of dorsolateral prefrontal cortex, ventrolateral prefrontal cortex, parietal lobule.
Example 19. The method of any one of examples 1 to 14, wherein said drug is Levodopa and wherein said treatment protocol is used to treat PD.
Example 20. The method of example 19, wherein said Levodopa dosage is in the range of 50-6000 mg.
Example 21. The method of examples 19 or 20, wherein said activation protocol increases the activation level of at least one brain region, selected from the list of: substantia nigra, caudate nucleus, putamen.
Example 22. The method of examples 19 or 20, wherein said activation protocol decreases the activation level of at least one brain region, selected from the list of: substantia nigra, caudate nucleus, putamen.
Example 23. The method of any one of examples 1 to 14, wherein said drug is Haloperidol and wherein said treatment protocol is used to treat Schizophrenia.
Example 24. The method of example 23, wherein said Haloperidol dosage is in the range of 1-10 mg.
Example 25. The method of examples 23 or 24, wherein said activation protocol increases the activation level of at least one brain region, selected from the list of: dorsolateral prefrontal cortex, dorsomedial prefrontal cortex, ventrolateral prefrontal cortex.
Example 26. The method of examples 23 or 24, wherein said activation protocol decreases the activation level of at least one brain region, selected from the list of: dorsolateral prefrontal cortex, dorsomedial prefrontal cortex, ventrolateral prefrontal cortex.
Example 27. A method for treating a neurological disease, comprising:

administering a drug according to a treatment protocol;

applying an activation protocol in a timed relationship to said administering, for selectively activation of at least one selected brain region;

wherein said selectively activation allows said drug to selectively interact with said at least one selected brain region.
Example 28. The method of example 27, wherein said applying comprises applying said activation protocol prior to said administering.
Example 29. The method according to example 27, wherein said applying comprises applying said activation protocol after said administering.
Example 30. The method of any one of examples 27 to 29, further comprising determining a brain activation profile prior to said applying.
Example 31. The method of any one of examples 27 to 30, further comprising determining a brain activation profile following said applying.
Example 32. The method of examples 30 or 31, further comprising modifying said activation protocol according to said determining.
Example 33. The method of any one of examples 30 to 32, further comprising modifying said drug dosage according to said determining.
Example 34. The method of any one of examples 27 to 33, further comprising determining the effect of said drug by measuring at least one clinical parameter value, following said applying.
Example 35. The method of example 34, wherein said clinical parameter is selected from a group comprising skin conductance, heart rate, blood pressure or blood flow, pupil diameter.
Example 36. The method of examples 34 or 35, further comprising modifying said activation protocol if said effect of said drug is not a desired effect.
Example 37. The method of any one of examples 27 to 36, wherein said timed relationship is adjusted according to said drug.
Example 38. The method of any one of examples 34 to 37, wherein said timed relationship is adjusted according to said effect.
Example 39. The method of any one of examples 27 to 38, wherein said timed relationship is at least 5 minutes before or after said applying.
Example 40. The method of any one of examples 34 to 39, wherein said effect comprises reducing at least one side effect of said drug.
Example 41. The method of any one of example s 27 to 40, wherein said drug is methylphenidate and wherein said neurological disease is ADHD.
Example 42. The method of example 41, wherein said methylphenidate dosage is in the range of 0.5-30 mg.
Example 43. The method of examples 41 or 42, wherein said activation protocol increases the activation level of at least one brain region selected from the list of: right/left dorsolateral prefrontal cortex ventrolateral prefrontal cortex, parietal lobule.
Example 44. The method of examples 41 or 42, wherein said activation protocol decreases the activation level of at least one brain region, selected from the list of dorsolateral prefrontal cortex, ventrolateral prefrontal cortex, parietal lobule.
Example 45. The method of any one of examples 27 to 40, wherein said drug is Levodopa and wherein said neurological disease is PD.
Example 46. The method of example 45, wherein said Levodopa dosage is in the range of 50-6000 mg.
Example 47. The method of examples 45 or 46, wherein said activation protocol increases the activation level of at least one brain region, selected from the list of: substantia nigra, caudate nucleus, putamen.
Example 48. The method of examples 45 or 46, wherein said activation protocol decreases the activation level of at least one brain region, selected from the list of: substantia nigra, caudate nucleus, putamen.
Example 49. The method of any one of examples 27 to 40, wherein said drug is Haloperidol and wherein said neurological disease is Schizophrenia.
Example 50. The method of example 49, wherein said Haloperidol dosage is in the range of 1-10 mg.
Example 51. The method of examples 49 or 50, wherein said activation protocol increases the activation level of at least one brain region, selected from the list of: dorsolateral prefrontal cortex, dorsomedial prefrontal cortex, ventrolateral prefrontal cortex.
Example 52. The method of examples 49 or 50, wherein said activation protocol decreases the activation level of at least one brain region, selected from the list of: dorsolateral prefrontal cortex, dorsomedial prefrontal cortex, ventrolateral prefrontal cortex.
Example 53. A drug in combination with an activation protocol for the treatment of a neurological disease.
Example 54. The drug according to example 53, wherein said drug is methylphenidate and wherein said neurological disease is ADHD.
Example 55. The drug according to example 54, wherein said activation protocol is applied at least 1 minute after or prior to the administration of said methylphenidate.
Example 56. The drug according to examples 54 or 55, wherein said activation protocol duration is at least 2 minutes.
Example 57. The drug according to any one of examples 54 to 56, wherein dosage of said methylphenidate is in the range of 0.5-30 mg.
Example 58. The drug according to any one of examples 54 to 57, wherein said activation protocol increases the activation level of at least one brain region selected from the list of: right/left dorsolateral prefrontal cortex ventrolateral prefrontal cortex, parietal lobule.
Example 59. The drug according to any one of examples 54 to 57, wherein said activation protocol decreases the activation level of at least one brain region, selected from the list of dorsolateral prefrontal cortex, ventrolateral prefrontal cortex, parietal lobule.
Example 60. The drug according to example 53, wherein said drug is Levodopa and wherein said neurological disease is PD.
Example 61. The drug according to example 60, wherein said activation protocol is applied at least 1 minute after or prior to the administration of said Levodopa.
Example 62. The drug according to examples 60 or 61, wherein said activation protocol duration is at least 2 minutes.
Example 63. The drug according to any one of examples 60 to 62, wherein said Levodopa dosage is in the range of 50-6000 mg.
Example 64. The drug according to any one of examples 60 to 63, wherein said activation protocol increases the activation level of at least one brain region, selected from the list of: substantia nigra, caudate nucleus, putamen.
Example 65. The drug according to any one of examples 60 to 63, wherein said activation protocol decreases the activation level of at least one brain region, selected from the list of: substantia nigra, caudate nucleus, putamen.
Example 66. The drug according to example 53, wherein said drug is Haloperidol and wherein said neurological disease is Schizophrenia.
Example 67. The drug according to example 66, wherein said activation protocol is applied at least 1 minute after or prior to the administration of said Haloperidol.
Example 68. The drug according to examples 66 or 67, wherein said activation protocol duration is at least 2 minutes.
Example 69. The drug according to any one of examples 66 to 68, wherein said Haloperidol dosage is in the range of 1-10 mg.
Example 70. The drug according to any one of examples 66 to 69, wherein said activation protocol increases the activation level of at least one brain region, selected from the list of: dorsolateral prefrontal cortex, dorsomedial prefrontal cortex, ventrolateral prefrontal cortex.
Example 71. The drug according to any one of examples 66 to 69, wherein said activation protocol decreases the activation level of at least one brain region, selected from the list of: dorsolateral prefrontal cortex, dorsomedial prefrontal cortex, ventrolateral prefrontal cortex.
Example 72. The drug according to any one of examples 53 to 71, wherein said drug is a psychotropic drug.
Example 73. A method for treating a neurological disease, comprising:

inhaling hyperbaric gas according to a hyperbaric treatment protocol;

applying an activation protocol in a timed relationship to said inhaling, for selectively activation of at least one selected brain region;

wherein said selectively activation allows said hyperbaric gas to selectively interact with said at least one selected brain region.
Example 74. The method of example 73, wherein said applying comprises applying said activation protocol during said inhaling.
Example 75. The method of examples 73 or 74, wherein said hyperbaric gas comprises oxygen or oxygen compounds.
Example 76. The method of examples 73 or 74, wherein said hyperbaric gas comprises nitrogen or nitrogen compounds.
Example 77. The method according to anyone of examples 73 to 76, wherein said neurological disease comprises stroke or TBI, and wherein said activation protocol selectively activates at least one damaged brain region.
Example 78. The method of example 1, wherein said administering further comprises inhaling said drug.
Example 79. The method of example 1, wherein said administering further comprises inhaling said drug.

Following are some additional examples of some embodiments of the invention:
Example 1. A method for modifying the effect of a drug by application of an activation protocol, comprising:

administering a drug according to a treatment protocol;

applying an activation protocol in a timed relationship to said administering, for differentially activation of at least one selected brain region;

wherein said differentially activation allows said drug to selectively interact with said at least one selected brain region.
Example 2. The method of example 1, wherein said applying further comprises applying an activation protocol before said administering.
Example 3. The method of example 1, wherein said applying further comprises applying an activation protocol after said administering.
Example 4. The method of any of the previous examples, further comprising determining a brain activation profile prior to said applying.
Example 5. The method of any of the previous examples, further comprising determining a brain activation profile following said applying.
Example 6. The method of examples 4 or 5, further comprising modifying said activation protocol according to said determining.
Example 7. The method of example 6 wherein said modifying comprises modifying said activation protocol to reach desired activation levels of at least one specific region and/or desired connectivity measures of at least one neural network by explicit or covert neurofeedback.
Example 8. The method of any one of examples 4 to 7, further comprising modifying said drug dosage according to said determining.
Example 9. The method of any of the previous examples, further comprising determining the effect of said drug by measuring at least one clinical parameter value, following said applying.
Example 10. The method of example 9, wherein said clinical parameter is selected from a group comprising skin conductance, heart rate, blood pressure or blood flow, pupil diameter.
Example 11. The method of examples 9 or 10, further comprising modifying said activation protocol if said effect of said drug is not a desired effect.
Example 12. The method of example 1, wherein said timed relationship is adjusted according to said drug.
Example 13. The method of example 5, wherein said timed relationship is adjusted according to said effect.
Example 14. The method of example 1, wherein said timed relationship is at least 5 minutes before or after said applying.
Example 15. The method of example 9, wherein said effect comprises reducing at least one side effect of said drug.
Example 16. The method of any of the previous examples, wherein said drug is methylphenidate and wherein said treatment protocol is used to treat ADHD.
Example 17. The method of example 16, wherein said methylphenidate dosage is in the range of 0.5-30 mg.
Example 18. The method of examples 16 or 17, wherein said activation protocol increases the activation level of at least one brain region selected from the list of: right/left dorsolateral prefrontal cortex ventrolateral prefrontal cortex, parietal lobule, striatum.
Example 19. The method of any one of examples 16 to 18, wherein said activation protocol decreases the activation level of at least one brain region, selected from the list of dorsolateral prefrontal cortex, ventrolateral prefrontal cortex, parietal lobule.
Example 20. The method of any one of examples 1 to 15, wherein said drug is Levodopa and wherein said treatment protocol is used to treat PD.
Example 21. The method of example 20, wherein said Levodopa dosage is in the range of 50-6000 mg.
Example 22. The method of examples 20 or 21, wherein said activation protocol increases the activation level of at least one brain region, selected from the list of: substantia nigra, caudate nucleus, putamen.
Example 23. The method of examples 20 or 21, wherein said activation protocol decreases the activation level of at least one brain region, selected from the list of: substantia nigra, caudate nucleus, putamen.
Example 24. The method of any one of examples 1 to 15, wherein said drug is Haloperidol and wherein said treatment protocol is used to treat Schizophrenia.
Example 25. The method of example 24, wherein said Haloperidol dosage is in the range of 1-10 mg.
Example 26. The method of examples 24 or 25, wherein said activation protocol increases the activation level of at least one brain region, selected from the list of: dorsolateral prefrontal cortex, dorsomedial prefrontal cortex, ventrolateral prefrontal cortex, striatum.
Example 27. The method of examples 24 or 25, wherein said activation protocol decreases the activation level of at least one brain region, selected from the list of: dorsolateral prefrontal cortex, dorsomedial prefrontal cortex, ventrolateral prefrontal cortex, striatum.
Example 28. The method of any one of the previous examples, wherein said applying an activation protocol comprises performing a neurofeedback protocol.
Example 29. The method of any one of the previous examples, wherein said activation protocol comprises a physical and/or a cognitive task.
Example 30. The method of any one of the previous examples, wherein said applying an activation protocol comprises performing at least one executive function task and/or at least one control inhibition task and/or at least one action planning paradigm.
Example 31. The method of any one of the previous examples, wherein said applying an activation protocol comprises performing at least one memory task and/or an interactive game.
Example 32. The method of any one of the previous examples, wherein said applying an activation protocol comprises applying said activation protocol using virtual reality.
Example 33. A method for treating a neurological disease, comprising:

administering a drug according to a treatment protocol;

applying an activation protocol in a timed relationship to said administering, for differentially activation of at least one selected brain region;

wherein said differentially activation allows said drug to selectively interact with said at least one selected brain region.
Example 34. The method of example 33, wherein said applying comprises applying said activation protocol prior to said administering.
Example 35. The method according to example 33, wherein said applying comprises applying said activation protocol after said administering.
Example 36. The method of any one of examples 33 to 35, further comprising determining a brain activation profile prior to said applying.
Example 37. The method of any one of examples 33 to 36, further comprising determining a brain activation profile following said applying.
Example 38. The method of examples 36 or 37, further comprising modifying said activation protocol according to said determining.
Example 39. The method of any one of examples 36 to 38, further comprising modifying said drug dosage according to said determining.
Example 40. The method of any one of examples 33 to 39, further comprising determining the effect of said drug by measuring at least one clinical parameter value, following said applying.
Example 41. The method of example 40, wherein said clinical parameter is selected from a group comprising skin conductance, heart rate, blood pressure or blood flow, pupil diameter.
Example 42. The method of examples 40 or 41, further comprising modifying said activation protocol if said effect of said drug is not a desired effect.
Example 43. The method of any one of examples 33 to 42, wherein said timed relationship is adjusted according to said drug.
Example 44. The method of any one of examples 40 to 43, wherein said timed relationship is adjusted according to said effect.
Example 45. The method of any one of examples 33 to 44, wherein said timed relationship is at least 5 minutes before or after said applying.
Example 46. The method of any one of examples 40 to 45, wherein said effect comprises reducing at least one side effect of said drug.
Example 47. The method of any one of examples 33 to 46, wherein said drug is methylphenidate and wherein said neurological disease is ADHD.
Example 48. The method of example 47, wherein said methylphenidate dosage is in the range of 0.5-30 mg.
Example 49. The method of examples 47 or 48, wherein said activation protocol increases the activation level of at least one brain region selected from the list of: right/left dorsolateral prefrontal cortex ventrolateral prefrontal cortex, parietal lobule.
Example 50. The method of examples 47 or 48, wherein said activation protocol decreases the activation level of at least one brain region, selected from the list of dorsolateral prefrontal cortex, ventrolateral prefrontal cortex, parietal lobule.
Example 51. The method of any one of examples 33 to 46, wherein said drug is Levodopa and wherein said neurological disease is PD.
Example 52. The method of example 51, wherein said Levodopa dosage is in the range of 50-6000 mg.
Example 53. The method of examples 51 or 52, wherein said activation protocol increases the activation level of at least one brain region, selected from the list of: substantia nigra, caudate nucleus, putamen.
Example 54. The method of examples 51 or 52, wherein said activation protocol decreases the activation level of at least one brain region, selected from the list of: substantia nigra, caudate nucleus, putamen.
Example 55. The method of any one of examples 33 to 46, wherein said drug is Haloperidol and wherein said neurological disease is Schizophrenia.
Example 56. The method of example 55, wherein said Haloperidol dosage is in the range of 1-10 mg.
Example 57. The method of examples 55 or 56, wherein said activation protocol increases the activation level of at least one brain region, selected from the list of: dorsolateral prefrontal cortex, dorsomedial prefrontal cortex, ventrolateral prefrontal cortex.
Example 58. The method of examples 55 or 56, wherein said activation protocol decreases the activation level of at least one brain region, selected from the list of: dorsolateral prefrontal cortex, dorsomedial prefrontal cortex, ventrolateral prefrontal cortex.
Example 59. A drug for use in the treatment of a neurological disease, wherein said drug is administered in a timed relationship to application of an activation protocol for differentially activation of at least one selected brain region.
Example 60. The drug according to example 59, wherein said drug is Venlafaxine or Tiagabine and said neurological disease is ADHD; or wherein said drug is Citalopram and said neurological disease is PTSD; or wherein said drug is selected from a list of Clomipramine, Tiagabine, Bupropion and Methylphenidate and said neurological disease is PTSD; or wherein said drug is selected from a list of Citalopram, Sertraline, Clomipramine and Venlafaxine and said neurological disease is OCD; or wherein said drug is selected from a list of Fluoxetine, Trazodone, N-arachidonoylaminophenol, and Risperadal and said neurological disease is Schizophrenia; or wherein said drug is selected from a list of Bupropion, Methylphenidate, and Venlafaxine and said neurological disease is Social anxiety; or wherein said drug is selected from a list of Milnacipran, Cannabis, Nabilone and Bupropion and said neurological disease is Chronic pain; or wherein said drug is selected from a list of Bupropion, Fluoxetine, Venlafaxine and Methylphenidate and said neurological disease is Addiction; or wherein said drug is Amphetamine and said neurological disease is Narcolepsy; or wherein said drug is Roboxetine and said neurological disease is Mild cognitive impairment; or wherein said drug is selected from a list of Methylphenidate, Ketamine, Bupropion, and Venlafaxine and said neurological disease is Depression; or wherein said drug is Apomorphine or Levodopa and said neurological disease is Movement disturbances; or wherein said drug is Valproic acid or Ibuprofen and said neurological disease is Epilepsy.
Example 61. The drug according to example 59, wherein said drug is methylphenidate and wherein said neurological disease is ADHD.
Example 62. The drug according to example 61, wherein said activation protocol is applied at least 1 minute after or prior to the administration of said methylphenidate.
Example 63. The drug according to examples 61 or 62, wherein said activation protocol duration is at least 2 minutes.
Example 64. The drug according to any one of examples 61 to 63, wherein dosage of said methylphenidate is in the range of 0.5-30 mg.
Example 65. The drug according to any one of examples 61 to 64, wherein said activation protocol increases the activation level of at least one brain region selected from the list of: right/left dorsolateral prefrontal cortex ventrolateral prefrontal cortex, parietal lobule.
Example 66. The drug according to any one of examples 61 to 64, wherein said activation protocol decreases the activation level of at least one brain region, selected from the list of dorsolateral prefrontal cortex, ventrolateral prefrontal cortex, parietal lobule.
Example 67. The drug according to example 59, wherein said drug is Levodopa and wherein said neurological disease is PD.
Example 68. The drug according to example 67, wherein said activation protocol is applied at least 1 minute after or prior to the administration of said Levodopa.
Example 69. The drug according to examples 67 or 68, wherein said activation protocol duration is at least 2 minutes.
Example 70. The drug according to any one of examples 67 to 69, wherein said Levodopa dosage is in the range of 50-6000 mg.
Example 71. The drug according to any one of examples 67 to 70, wherein said activation protocol increases the activation level of at least one brain region, selected from the list of: substantia nigra, caudate nucleus, putamen.
Example 72. The drug according to any one of examples 67 to 70, wherein said activation protocol decreases the activation level of at least one brain region, selected from the list of: substantia nigra, caudate nucleus, putamen.
Example 73. The drug according to example 59, wherein said drug is Haloperidol and wherein said neurological disease is Schizophrenia.
Example 74. The drug according to example 73, wherein said activation protocol is applied at least 1 minute after or prior to the administration of said Haloperidol.
Example 75. The drug according to examples 73 or 74, wherein said activation protocol duration is at least 2 minutes.
Example 76. The drug according to any one of examples 73 to 75, wherein said Haloperidol dosage is in the range of 1-10 mg.
Example 77. The drug according to any one of examples 73 to 76, wherein said activation protocol increases the activation level of at least one brain region, selected from the list of: dorsolateral prefrontal cortex, dorsomedial prefrontal cortex, ventrolateral prefrontal cortex.
Example 78. The drug according to any one of examples 73 to 76, wherein said activation protocol decreases the activation level of at least one brain region, selected from the list of: dorsolateral prefrontal cortex, dorsomedial prefrontal cortex, ventrolateral prefrontal cortex.
Example 79. The drug according to any one of examples 59 to 78, wherein said drug is a psychotropic drug.
Example 80. A device for delivery of an activation protocol, comprising:

a memory, wherein said memory stores indications related to at least one drug and at least one activation protocol;

an interface configured to deliver at least one human detectable indication and said activation protocol;

a control circuitry, wherein said control circuitry signals said interface to deliver said at least one human detectable indication and said activation protocol based on said indications stored in said memory.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product.

Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave.

Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as activation of brain regions, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

Table A is a table depicting examples of the conditions (clinical diagnosis), mental indications, candidate brain targets, drugs/compounds, pathological subclasses of the drugs/compounds, biochemical subclasses of the drugs/compounds, mechanism of each drug/compound, range of daily dosage for each drug/compound in milligram, of the drug and mechanisms, according to some embodiments of the invention;

FIG. 1A is a block diagram depicting the main components of a system for enhancement of drug effect, according to some embodiments of the invention;

FIG. 1B is a general flow chart depicting a process for drug effect enhancement, according to some embodiments of the invention;

FIG. 2 is a block diagram depicting the relation between a drug, an activation protocol and a brain activation profile, according to some embodiments of the invention;

FIG. 3A is a general flow chart depicting a process for using a device for applying an activation protocol, according to some embodiments of the invention;

FIGS. 3B-3G are graphs depicting the pharmacokinetics and pharmacodynamics of a drug following an activation protocol, according to some embodiments of the invention;

FIG. 3H is a flow chart depicting a process for application of an activation protocol using neurofeedback and/or virtual reality techniques, according to some embodiments of the invention;

FIG. 4 is a flow chart depicting a process for matching an activation treatment to a desired activation profile, according to some embodiments of the invention;

FIGS. 5A-5C are flow charts depicting a detailed process for using a device for application of an activation protocol, according to some embodiments of the invention;

FIGS. 5D-5E are block diagrams depicting the components of a device for drug effect enhancement, according to some embodiments of the invention;

FIG. 5F is an illustration depicting a system for enhancement of drug effect, according to some embodiments of the invention;

FIGS. 6A-6D describe the design and the results of a validation experiment when comparing the coupling of a cognitive task with a drug or with a placebo, according to some embodiments of the invention;

FIGS. 7A-7B describe the design and the results of a validation experiment, comparing the coupling of a cognitive task with a varying dose of a drug, according to some embodiments of the invention; and

FIGS. 8A-8H describe the results of a validation experiment for measuring neurobehavioral effects of Methylphenidate and right inferior frontal gyms neurofeedback activation combined treatment for ADHD, according to some embodiments of the invention.

### DESCRIPTION OF SPECIFIC EMBODIMENTS OF THE INVENTION

The present invention, in some embodiments thereof, relates to a method for modifying the effect of a drug and, more particularly, but not exclusively, to a method for modifying the effect of psychotropic drugs.

An aspect of some embodiments relates to modifying the effect of a drug by affecting at least one brain region. In some embodiments, the brain region is affected before and/or after the administration of the drug, for example using an activation protocol. In some embodiments, the activation protocol affecting the at least one brain region increases the therapeutic effect of the drug, for example by modulating the availability of drug molecules in the at least one brain region and/or in other brain regions. Alternatively, the activation protocol affecting the at least one brain region decreases the therapeutic effect of the drug, for example, by reducing the availability of drug molecules in at least one brain region and/or in other brain regions. Optionally, the activation protocol activates at least one brain region before or after the administration of a drug for example to reduce at least one side effect of the drug.

In some embodiments, an activation protocol is a physical and/or a cognitive task, which is not chemically induced. In some embodiments, application of an activation protocol selectively affects at least one selected brain region, optionally by increasing or reducing the activity of neurons or other neural cell types such as glia cells in that region. In some embodiments, increasing the activity of cells in a selected region leads to an increase in blood flow to this region. In some embodiments, by increasing the blood flow to a selected brain region, the bioavailability of a drug, for example a psychotropic drug, in that selected brain region, is also increased. In some embodiments of the invention, the drug being used is a psychotropic drug which is a chemical compound that changes brain function and results in alterations in perception and/or mood and/or consciousness. Optionally, activation of a selected brain region reduces at least one side-effect of the drug, for example by minimizing the bioavailability of the drug in undesirable regions of the brain. In some embodiments, the activation protocol is determined according to a desired activation of at least one brain region or a neuronal network. Alternatively or additionally, the activation protocol is determined according to at least one desired effect of the drug. Optionally, the activation protocol is determined according to the pharmacokinetic and/or pharmacodynamics properties of the drug.

In some embodiments, application of an activation protocol before and/or after the administration of the drug allows to, for example to administer a lower dose of the drug in order to reach a desired effect. Optionally, lowering the drug dosage allows to for example, to minimize undesired side effects of the drug. Alternatively or additionally, application of an activation protocol before and/or after the administration of a drug reduces the time needed to reach a desired effect of the drug.

In some embodiments, the drug belongs to at least one of three drug families, reuptake inhibitors drugs for example Ritalin®, agonist drugs for example Levodopa or antagonistic drugs for example Haloperidol. In some embodiments, Ritalin® is used for treating attention deficit hyperactivity disorder (ADHD), for example by blocking dopamine and norepinephrine transporters. In some embodiments, Levodopa is used for treating Parkinson's disease (PD). In some embodiments, Levodopa is converted into Dopamine and replaces the endogenous dopamine molecules in the central nervous system (CNS). In some embodiments, Haloperidol is used for the treatment of Schizophrenia, for example, by blocking dopaminergic receptors.

A method for affecting one or more brain regions, for example using an activation protocol, is based on an endogenous mechanism which may couple vascular changes with behaviorally-induced neural responses. These responses may correspond to increased cerebral blood flow, and may thus enhance local availability of pharmacological agents. In some embodiments, the neurovascular coupling mechanism leads, for example, to functional hyperemia. Functional hyperemia may comprise increasing blood supply to regions with neuronal activation. Enhancing the efficacy of a drug is probably achieved by synchronizing this neurovascular coupling process with the administration of a drug related to one of the abovementioned drug families.

In some embodiments, the method may enhance a pharmacological therapeutic effect of a drug and/or diminishing an adverse side effect associated with the drug, for example, in a subject who has been treated with the drug or who is to be administered with the drug. The method comprises, for example, structured protocols for psychological stimulation, which may be tightly timed according to the drug's pharmacokinetic and/or pharmacodynamic characteristics. In some embodiments, the protocols may comprise neurofeedback protocols in which the subject modulates, for example, the activity level of brain regions of interest on the basis of a continuous feedback on the activation of the target region. In these cases, the neurofeedback is based, for example, on functional Magnetic Resonance Imaging (fMRI) and/or functional Near-Infrared Spectroscopy (fNIRS), and/or electroencephalography (EEG; especially scalp EEG fingerprint of local activity as measured with higher spatial resolution such as fMRI and intracranial recording). The monitoring of the target brain regions may cue drug administration so that the drug fraction in the target brain region will be highest when the blood supply to the target region culminates.

The enhancement of a pharmacological therapeutic effect of a drug may refer to the enhancement of at least one measurable or observable clinical therapeutic parameter (characteristic) of the mental and/or physiological state (treated, prevented or enhanced) by the drug. The enhancement may be, for example, in the reduction or elimination or prevention of a disease, symptoms of the disease, and/or side effects of a disease in the subject who has been treated with said drug or who is to be guided with said procedure.

Optionally or additionally, enhancement may be in a psychophysiological function of non-patients that is improved by a certain drug.

Without being necessarily bound by theory, the two major factors that play a key role in mediating the pharmacologic-functional effects are most probably the cerebral blood volume (CBV) and the cerebral blood flow (CBF). The total cerebral blood volume (i.e., across the different types of blood vessels) may be increased in about 20%, following neural activation. Additionally, animal, human, and simulation studies estimated that capillary blood volume increases during functional stimulation in about 10.5%-17% (e.g., Chen and Pike, 2009; Ciris et al., 2014; Krieger et al., 2012; Stefanovic et al., 2008).

Functional stimulation may also affect the cerebral blood flow. The flow-volume power law relationship in humans is estimated as ΔCBV=ΔCBF0.23 on average, and CBF is increased in about 47%-60% following high intensity visual and sensorimotor stimulations in humans (Chen and Pike, 2009; Ciris et al., 2014).

An estimation of the Functional Pharmacological Coupling (FPC) on the momentary influx of the drug from the plasma to the brain (Kᵢₙ) can be derived from the Renkin-Crone equation of capillary transport (Bickel, 2005; Crone, 1963; Renkin, 1959) under a few assumptions:

$${K}_{in} = F ( 1 - {e}^{- \frac{PS}{F}} ) \qquad ( 1 )$$

where F is the cerebral blood flow (CBF) velocity, P is the permeability constant, and S is the capillary surface area. In some embodiments, the influx gain following the FPC will depend on Sₒ and Fₒ (the surface area and flow rate before stimulation) as follows:

$$Δ {K}_{in} = \frac{{F}_{c} ( 1 - {e}^{- \frac{{PS}_{0}}{{F}_{0}} \frac{\sqrt{{V}_{c}}}{{F}_{c}}} )}{1 - {e}^{- \frac{{PS}_{0}}{{F}_{0}}}} \qquad ( 2 )$$

where V_{c} and F_{c} are factors of the capillary blood volume and blood flow change following functional stimulation, respectively. In some embodiments, with high permeability constants, ΔKᵢₙ will approach F_{c}; i.e., an FPC effect of 47%-60%.

It should be noted that this estimation relies on a model, which does not take into account the efflux rate (i.e., brain-to-plasma flow), tissue binding, local metabolism, and clearance by the interstitial fluid flow. Additional parameters that may interact with the FPC effect comprise potential change in BBB permeability following activation, and the enhancement of vesicle formation in the BBB following an increase in the ligand availability to membrane receptors.

FPC may be particularly advantageous in cases in which an increase in drug doses is not linearly translated into higher drug fraction in the target tissue due to “bottlenecks” such as metabolic processing in the liver. Since the FPC effect takes place in situ (i.e., by redistributing the drug fraction that is already in the brain), it may produce a linear effect in spite of the metabolic bottleneck.

FPC may decrease adverse effects, for example, by reducing the local CBF in non-target regions. Therefore the functional stimuli in the FPC protocol most probably will be designed to facilitate not only the activation of the target regions, but also the deactivation of non-target regions.

Reference is now being made to Table A listing the conditions (clinical diagnosis), mental indications, candidate brain targets, drugs/compounds, pathological subclasses of the drugs/compounds, biochemical subclasses of the drugs/compounds, mechanism of each drug/compound, range of daily dosage for each drug/compound in milligram, of the drug and mechanisms, according to some embodiments of the invention. In addition, table A includes an example of an activation protocol task for each drug/compound in the list. In some embodiments, application of the listed activation protocol for each drug increases the bioavailability of the drug in the corresponding candidate brain target, as described herein.

Table A abbreviations: ACC—anterior cingulate cortex, ADHD—Attention deficit hyperactivity disorder, DA—dopamine, dLPFC—dorsolateral prefrontal cortex, dmPFC—dorsomedial prefrontal cortex, IFG—inferior frontal gyrus, NaCC—nucleus accumbens, OCD—obsessive compulsive disorder, OFC—orbitofrontal cortex, PFC—prefrontal cortex, PTSD—posttraumatic stress disorder, RAS—reticular activating system, sg—subgenual, SMA—Supplementary motor area, a—anterior, d—dorsal, r—right, m—medial, v—ventral. IR: immediate-release, SR: sustained-release, ER: extended-release, PO: per os.

According to some embodiments and without being bound by theory, attention deficit hyperactivity disorder (ADHD) is a condition in which an individual chronically manifests one of the following behaviors: not being able to focus (inattentiveness), being overactive (hyperactivity), or not being able control behavior (impulsivity).

According to some embodiments and without being bound by theory, post-traumatic stress disorder (PTSD) is a type of anxiety disorder following an extreme emotional and/or physical trauma that usually involves threat. PTSD symptoms include highly emotional and negatively episodes during which an individual of relives the traumatic event, avoidance from elements that are associated with the traumatic event, hypervigilance, and negative thoughts and mood or feelings.

According to some embodiments and without being bound by theory, social anxiety disorder is a condition in which an individual is persistently afraid and tends to avoid social situations in which he or she may be judged by other individuals so that his or her social functioning is considerably impaired.

According to some embodiments and without being bound by theory, obsessive-compulsive disorder (OCD) is a condition of repeated feelings, sensations, and thoughts, (obsessions), and an urge to perform repeatedly a behavior regardless of its effect and in a non-adaptive manner (compulsions).

According to some embodiments and without being bound by theory, schizophrenia is a condition of impaired distinction between reality and non-reality. Its symptoms include hallucinations, delusions, and intrusive thoughts (positive symptoms), blunting of affect, apathy, and anhedonia (negative symptoms).

According to some embodiments and without being bound by theory, chronic pain is often defined as any pain lasting more than three months. It may follow a physical injury, but in other cases it may have no clear organic source. Chronic pain may be accompanied with other adverse conditions including sleep disturbance and chronic fatigue, and mood changes, and decreased appetite.

According to some embodiments and without being bound by theory, addiction is a chronic compulsive need for a substance accompanied with a substantial psychological and physiological difficulty to abstain this substance. It is characterized by an increased tolerance to the substance and physiological symptoms upon withdrawal.

According to some embodiments and without being bound by theory, narcolepsy is a neurological disorder characterized by extreme sleepiness and attacks of daytime sleep.

According to some embodiments and without being bound by theory, mild cognitive impairment (MCI) involves minor deficits in cognitive abilities that do not significantly impact daily functioning. MCI patients may have impairments in memory, language, thinking and executive functions that are higher than normal age-related changes.

According to some embodiments and without being bound by theory, depression (major depressive disorder) is a common mood disorder that may involve a continuous loss of interest in human activities, a prolonged sense of sadness, worthless, hopelessness and emptiness, anhedonia, fatigue or lack of energy, self-isolation, thoughts of death and suicide attempts, aches, and sudden change in appetite.

According to some embodiments and without being bound by theory, movement disturbances involve impaired control over movement that is caused by neural disorder. Movement disturbances include tremor, dystonia (sustained or repetitive muscle contractions), Chorea (rapid, involuntary movement), hypokinesia (reduced amplitude of movements), and rigidity.

According to some embodiments and without being bound by theory, epilepsy is a condition of recurrent epileptic seizures, unprovoked by any immediate identified cause. Epileptic seizures are caused by a sudden abnormal excessive and synchronous neuronal discharge in the central nervous system. They are accompanied by transient changes in motor behavior, autonomic function, and consciousness.

According to some embodiments, the activation protocol, for example one or more of the activation protocols described in table A and/or in other parts of the application, is applied in a timed relationship, for example before, during and/or after the administration of a drug, for example one of the drugs listed in Table A. In some embodiments, the activation protocol application is initiated at least 1 minute after the administration of a drug, for example 1 minute, 2 minute, 5 minute, 10 minute or any intermediate, shorter or longer time after the administration of a drug. In some embodiments, the activation protocol application is initiated up to 1 hour after the administration of a drug, for example 60 minutes, 50 minutes, 30 minutes after the administration of the drug or any intermediate, shorter or longer period.

According to some embodiments, the activation protocol is applied according to a pharmacokinetic profile and/or a pharmacodynamics profile of the drug. In some embodiments, the activation protocol is applied in a timed relationship to a pharmacokinetic profile and/or a pharmacodynamics profile of the drug. In some embodiments, the application of the activation protocol initiates or ends when at least 10% of the drug enters into the brain, for example 10%, 20%, 30%, 40%, 50% or any intermediate, smaller or larger percentage. Alternatively or additionally, the application of the activation protocol initiates or ends at least 5 minutes prior to reaching the maximal effect of the drug in a selected brain region, for example the brain region being activated by the activation protocol. In some embodiments, the duration of the application protocol is in a range of 10 seconds to 40 minutes, for example, 10 seconds, 30 seconds, 1 minute, 5 minute, 10 minute, 20 minute or any intermediate, smaller or larger value.

According to some embodiments, the activation protocol is applied for differentially activation of at least one selected brain region. In some embodiments, differential activation of at least one selected brain region refers to a relative increase in the firing of a population of neurons in the selected brain region compared to other brain regions. Alternatively or additionally, differential activation of at least one selected brain region refers to an increase of up to 100% in local cerebral blood flow in the at least one selected brain region compared to other brain regions, for example an increase of 80%, an increase of 70%, an increase of 60%, an increase of 50% or any intermediate, smaller or larger increase in cerebral blood flow. Alternatively or additionally, differential activation of at least one selected brain region refers to up to 60% increase in the local blood volume in the selected brain region compared to other brain regions, for example an increase of 50%, an increase of 40%, an increase of 30%, an increase of 20% or any intermediate, smaller or larger increase in the local blood volume.

An aspect of some embodiments relates to applying an activation protocol together with a hyperbaric gas, for example to affect the uptake of the gas by a brain tissue. In some embodiments, the activation protocol is applied before, during or after the inhalation of the hyperbaric gas. In some embodiments, the activation protocol is applied before, during or after a hyperbaric medicine treatment session.

In some embodiments, the application of the activation protocol increases and/or decreases the uptake of the gas with at least one brain region. In some embodiments, the hyperbaric gas comprises hyperbaric atmospheric gas, or hyperbaric atmospheric gas compounds.

In some embodiments, the hyperbaric gas comprises hyperbaric oxygen gas or hyperbaric oxygen gas compounds. In some embodiments, the hyperbaric gas comprises hyperbaric nitrogen gas or hyperbaric nitrogen gas compounds.

Without being bound by theory, application of activation protocol to affect at least one brain region increase the flow of the gas into at least one brain region or tissue type. Additionally or alternatively, application of activation protocol directs an angiogenic effect of the hyperbaric gas to the activated brain region.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary System for Enhancing the Effect of a Psychotropic Drug

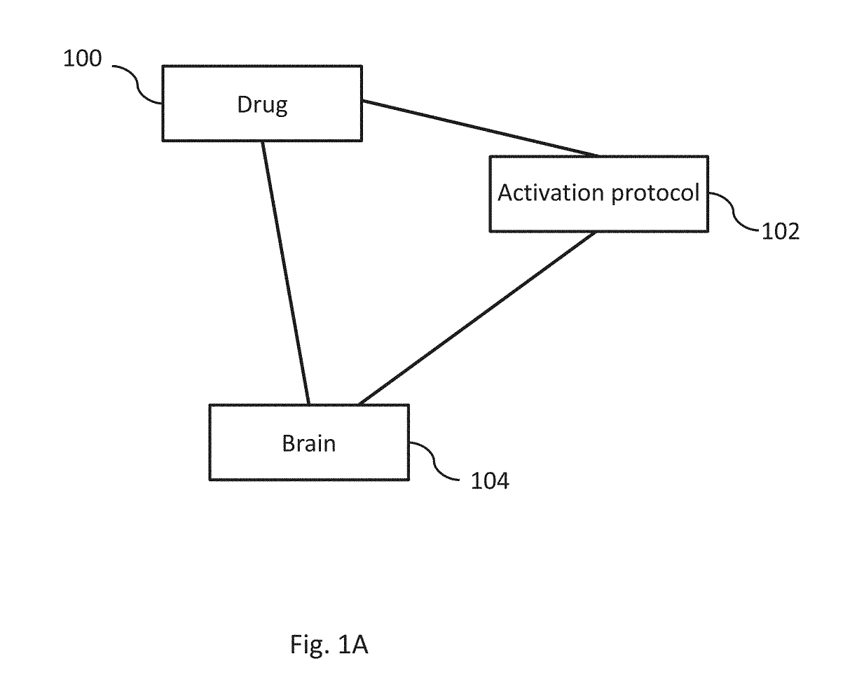

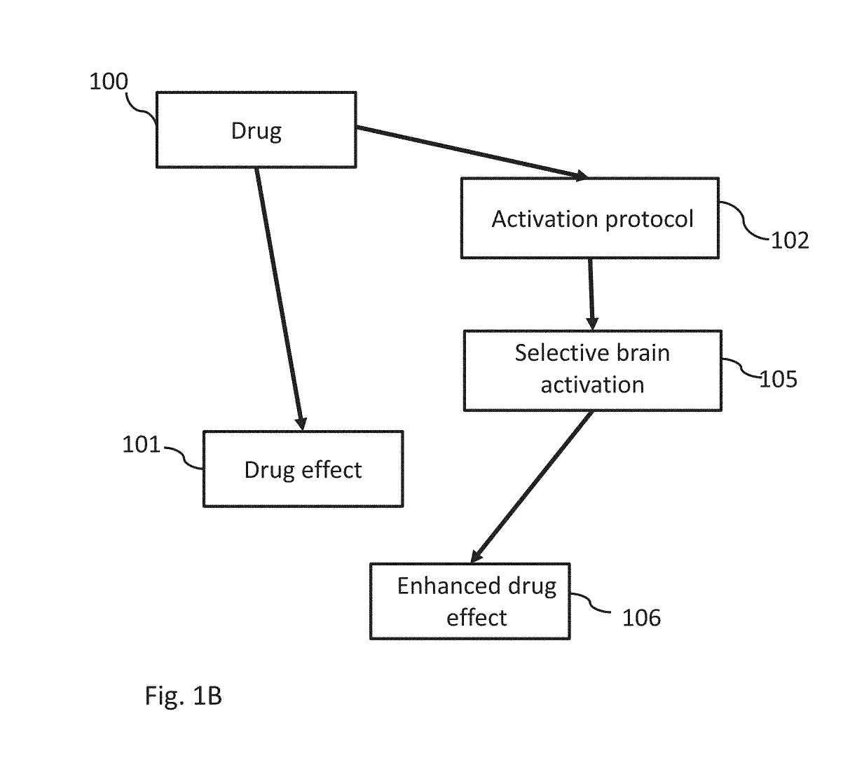

Reference is now made to FIG. 1A depicting the main components of a system for modifying the effect of a psychotropic drug, according to some embodiments of the invention.

According to some exemplary embodiments, drug 100, for example a psychotropic drug acts on specific regions of the brain 104 and/or on at least one neuronal network. In some embodiments, drug 100, for example Ritalin®, enhances working memory by optionally acting on the dorsolateral prefrontal cortex and/or the posterior parietal cortex. Alternatively, drug 100, for example Levodopa, acts on the nigrostriatal pathway. In some embodiments, Levodopa is administered in a dosage range of 50-6000 mg. Optionally, drug 100, for example haloperidol, acts on at least one of the frontal cortical regions of the brain. In some embodiments, Haloperidol is administered in a dosage range of 1-10 mg. Alternatively, drug 100 affects specific sub-populations of neurons, for example dopaminergic neurons and/or neural cells, for example glia cells.

In some embodiments, drug 100 is a reuptake inhibitor (RI) drug, for example Ritalin® (Methylphenidate) which is used for treating ADHD. In some embodiments RI drugs act by counteract a target neurotransmitter by directly competing with it on the binding to a membrane transporter and blocking. Alternatively, RI drugs indirectly counteract a target neurotransmitter by binding to allosteric sites. In some embodiments, when using RI drugs, the removal of the neurotransmitter from the extracellular space is reduced, its availability to the synaptic receptors increases and so is the neurotransmission.

In some embodiments, drug 100 is an agonist drug, for example Levodopa which is used for treating Parkinson's disease (PD). In some embodiments, agonist drugs compete with an endogenous ligand on binding and activating a receptor for example, Levodopa is converted to dopamine in the body and competes with endogenous dopamine on binding and activating dopaminergic receptors.

In some embodiments, drug 100 is an antagonistic drug, for example Haloperidol which is used for treating schizophrenia. In some embodiments, antagonistic drugs block the binding of an endogenous agonist to a receptor, which dampens the agonist-mediated response.

According to some exemplary embodiments, an activation protocol 102, for example a neurofeedback treatment increases the activation of at least one brain region in the brain and/or decreases the activation of at least one other brain region. Alternatively, activation protocol 102 increases the activation of at least one sub-population of neurons for example, dopaminergic neurons and/or decreases the activation of at least one sub-population of cells. Optionally, activation protocol 102 increases the activity of a selected neural network and/or decreases the activity of at least one neuronal network.

According to some exemplary embodiments, drug 100 is distributed throughout the body, and enters brain 104 through the blood flow. In some embodiments, drug 100 distribution in brain 104 is based on the blood supply to different regions of brain 104.

In some embodiments, controlling the blood supply to different brain regions allows, for example, to control the distribution of drug 100 throughout brain 104. In some embodiments, activation protocol 102 increases the blood supply to the activated brain regions and/or neuronal networks. Alternatively or additionally, activation protocol 102 increases the blood supply to activated neuronal cells. In some embodiments, increasing the blood supply to selective brain regions, leads for example, to an increase in the concentration of drug 100 in these selective brain regions.

Optionally, increasing the blood supply to selective brain regions, leads for example, to a decrease in drug 100 concentrations in less active brain regions.

Reference is now made to FIG. 1B, depicting a process for application of an activation protocol after administration of a drug, according to some embodiments of the invention.

According to some exemplary embodiments, a psychotropic drug, for example drug 100 is administered to treat a neurological condition. In some embodiments, drug 100 enters the brain and is distributed throughout different brain regions. In some embodiments, drug 100 induces drug effect 101 when drug 100 affects selected brain regions related to the neurological condition. Additionally, drug 100 is distributed in brain regions that are not related to the neurological condition, which may lead for example, to unwanted side effects.

According to some exemplary embodiments, an activation protocol 102, for example a cognitive task or a TMS treatment, or a transcranial direct current stimulation (tDCS), or a transcranial alternating current stimulation (tACS) or a direct stimulation is applied after the administration of drug 100. Alternatively, activation protocol 102 is applied before and/or during the administration of drug 100. In some embodiments, activation protocol 102 leads to selective brain activation 105 of at least one selected brain region. In some embodiments, the selected brain region is related to the neurological condition. In some embodiments, selective brain activation 105 leads to an increase in blood supply to the activated brain region. In some embodiments, the increase in blood supply to at least one brain region that is related to the neurological condition, increase the concentration of drug 100 in the activated brain region, which leads for example, to an enhanced drug effect 106.

In some embodiments, selective brain activation 105 comprises selective activation of at least one selected brain region. In some embodiments, selective activation of at least one selected brain region is determined by blood flow measurements in this selected region or by neuronal activation measurements, for example using EEG or functional MRI measurements. In some embodiments, when using EEG to measure neuronal activation in at least one brain region, the neuronal activation pattern and/or the synchronization level of neurons in that region is determined.

In some embodiments, activation protocol 102 is applied to activate at least one brain region that is not related to the neurological condition, for example to decrease the effect of drug 100 in brain regions related to the neurological condition.

According to some exemplary embodiments, application of activation protocol 102 before and/or during and/or after administration of drug 100, allows for example, to control the effect of drug 100 by affecting the distribution of drug 100 throughout selective brain regions. In some embodiments, application of activation protocol 102, allows to, for example to reduce the dosage of drug 100 that is required to reach a desired drug effect.

According to some exemplary embodiments, the drug 100 is MPH and the activation protocol 102 is selected to increase and/or decrease the activation of at least one brain region selected from a list of right/left dorsolateral prefrontal cortex ventrolateral prefrontal cortex, parietal lobule, and/or striatum.

According to some exemplary embodiments, the drug 100 is Haloperidol and the activation protocol 102 is selected to increase and/or decrease at least one brain region selected from a list of dorsolateral prefrontal cortex, dorsomedial prefrontal cortex, ventrolateral prefrontal cortex, striatum.

### Exemplary Activation Protocols

According to some exemplary embodiments, activation protocol 102 is selected to match drug 100, for example Ritalin® or Levodopa. Alternatively or additionally, the activation protocol 102 is selected according to drug 100 dosage. Optionally, the activation protocol is selected according to pharmacokinetics and/or pharmacodynamics of drug 100, for example according to the penetration rate of the drug through the BBB and/or according to the clearance rate of the drug from the blood.

In some embodiments, activation protocol 102 is selected according to the activation mechanism of drug 100 and/or according to the distribution of drug 100 in neural tissue. Alternatively or additionally, activation protocol 102 is selected according to the administration regime of the drug.

According to some embodiments, the activation protocol comprises application of at least one cognitive task, for example, viewing of dynamic emotional content (e.g., movies, music, and virtual reality) to optionally direct drugs to the amygdala.

Alternatively or additionally, the activation protocol comprises performing at least one executive function and/or control inhibition tasks (e.g., go-no go, N-back), for example to direct the drug to dorsolateral and/or ventrolateral prefrontal regions. In some embodiments, the activation protocol comprises performing at least one action planning paradigm, for example to direct drugs to the dorsal anterior cingulate cortex.

Alternatively or additionally, the activation protocol comprises performing at least one interactive game optionally with risk and/or punishment and/or reward, for example to direct the drug to the nucleus accumbens. In some embodiments, the activation protocol comprises performing at least one memory task, for example to direct the drug to the hippocampus.

According to some exemplary embodiments, the activation protocol comprises application of at least one physical task for example game-like scenario in virtual reality using treadmills or haptic devices, and motion capturing equipment to enhance the delivery of drugs such as Levodopa (L-DOPA) optionally across the nigrostriatal stream. In some embodiments, the cognitive and/or physical task is performed only once before or after the administration of the drug. Alternatively, the cognitive task is repeated at least 2 times before or after the administration of the drug.

According to some embodiments, when drug 100 comprises Ritalin®, activation protocol 102 includes visualizing at least one directed movement scenario, for example in a virtual-reality environment and/or using a handheld device. In some embodiments, in this scenario, the subject will is prompted to perform at least one motoric activity. In some embodiments, the activation protocol 102 for Levodopa lasts for 1-60 minutes, and optionally is be applied 1-30 minutes or 30-60 minutes after the administration of Levodopa. In some embodiments, the abovementioned activation protocol directs Levodopa to striatal locations in the brain, for example the caudate nucleus.

According to some embodiments, when drug 100 comprises Ritalin®, activation protocol 102 includes performing at least one task related to sustained attention and/or working memory and/or at least one arithmetic challenge, for example using a handheld device. In some embodiments, these tasks comprise N-back tasks, visual puzzles, and span tasks. In some embodiments, the activation protocol 102 for Ritalin® lasts for 1-60 minutes, for example 30-45 minutes. In some embodiments, the activation protocol is applied 1-80 minutes, for example 5-30 minutes or 30-60 minutes after administration of Ritalin®. Alternatively, the activation protocol is applied 1-10 minutes before Ritalin® administration. In some embodiments, application of activation protocol 102 directs Ritalin® to the inferior frontal gyrus and/or the dorsolateral prefrontal cortex and/or the parietal lobule.

According to some exemplary embodiments, the activation protocol is applied before or after administration of 0.5-30 mg of Ritalin®.

According to some embodiments, when drug 100 comprises Haloperidol, activation protocol 102 includes performing at least one task related to working memory, arithmetic challenges for example N-back tasks, visual puzzles and/or span tasks. Optionally, the activation protocol 102 comprises visualizing self-related and/or empathy-eliciting content, for example using a handheld device. In some embodiments, the activation protocol 102 for Haloperidol lasts for 1-60 minutes, for example 30-45 minutes. In some embodiments, the activation protocol is applied 1-80 minutes, for example 5-30 minutes or 30-60 minutes after administration of Haloperidol. In some embodiments, application of activation protocol 102 directs Haloperidol to at least one prefrontal region of the brain.

### Exemplary Activation Profile of a Drug

According to some exemplary embodiments, a drug is administered for a treatment of a neurological condition. In some embodiments, a desired activation profile of the drug includes activation of at least one selected brain region and/or a sub-class of neuronal cells located in the selected brain region. In some embodiments, the selected brain region and/or the sub-class of neuronal cells are related to the neurological condition. Optionally, the selected brain region and/or the sub-class of neuronal cells are related to at least one symptom of the neurological condition. In some embodiments, a desired activation profile of the drug includes reducing the effect of the drug in at least one selected brain region and/or in at least one sub-population of neuronal cells located in this brain region.

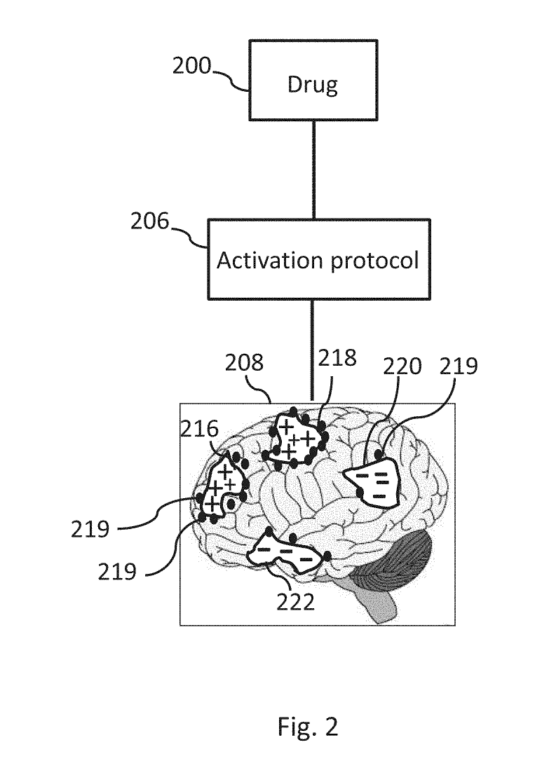

Reference is now made to FIG. 2 depicting an activation profile of a drug based on activation of at least one selective brain region, according to some embodiments of the invention.

According to some exemplary embodiments, an activation protocol 206 is applied after the administration of drug 200. In some embodiments, application of activation protocol 206 leads to, for example an increased activation of brain regions 216 and 218. Optionally, application of activation protocol 206 leads to, for example, a decreased activation of brain regions 220 and 222. In some embodiments, the blood supply to brain regions 216 and 218 is increased following the activation. In some embodiments, the blood supply to brain regions 220 and 222 is decreased following the activation

In some embodiments, drug molecules 219 travel in the blood stream and concentrate in or at the vicinity of activated brain regions, for example brain regions 216 and 218. In some embodiments, the concentration of drug molecules 219 is reduced in or at the vicinity of regions where blood supply is decreased, for example brain regions 220 and 222.

In some embodiments, the brain regions that are affected from the activation protocol are selected from a list of ventrolateral prefrontal cortex, parietal lobule, substantia nigra, caudate nucleus, putamen, dorsomedial prefrontal cortex, dorsolateral prefrontal cortex,

According to some exemplary embodiments, each drug, for example psychotropic drug, has its own desired activation profile. In some embodiments, the desired activation profile of a drug is determined by its composition, and/or dosage and/or structure and/or pharmacokinetic properties. In some embodiments, a desired activation profile of a drug is based, for example, on at least one desired brain target for the drug. In some embodiments, an activation protocol is selected and/or adjusted for each drug and/or to induce, for example, a desired activation profile of the drug.

### Exemplary Process for Application of an Activation Protocol

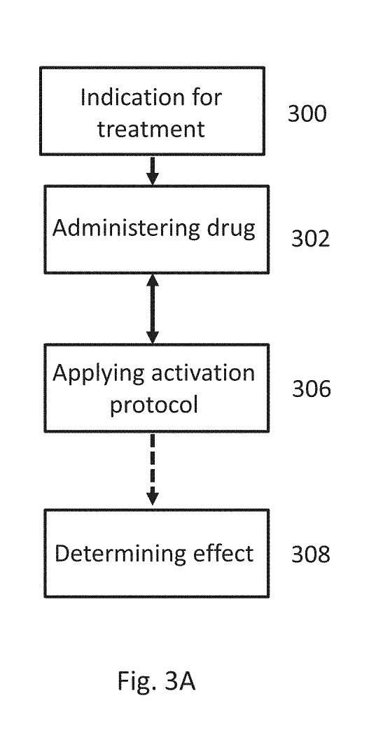

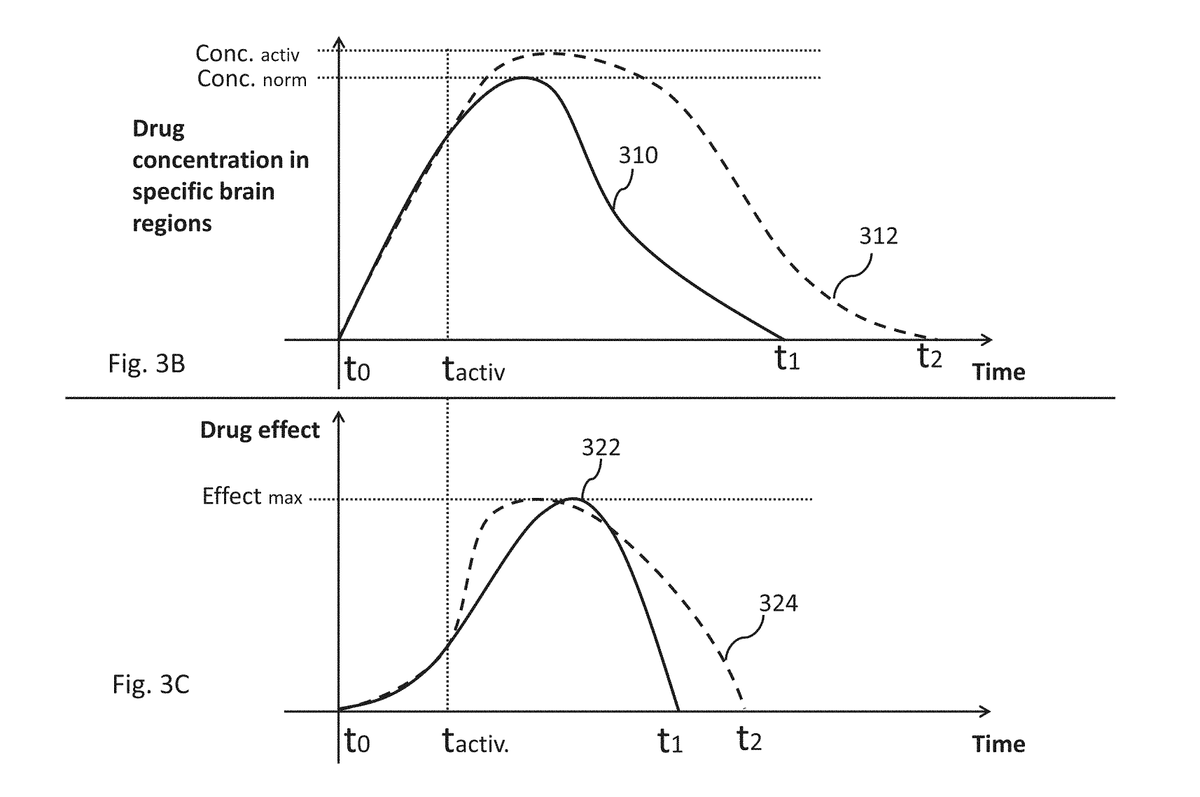

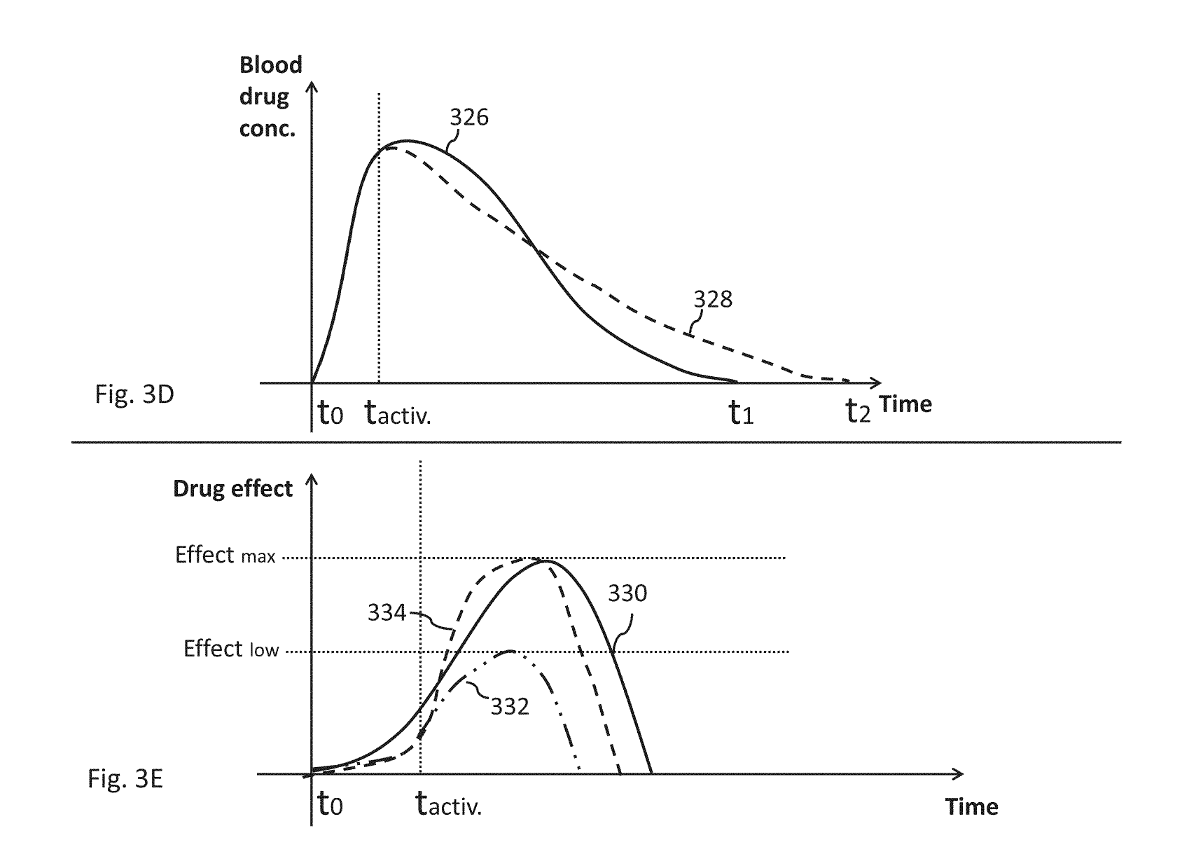

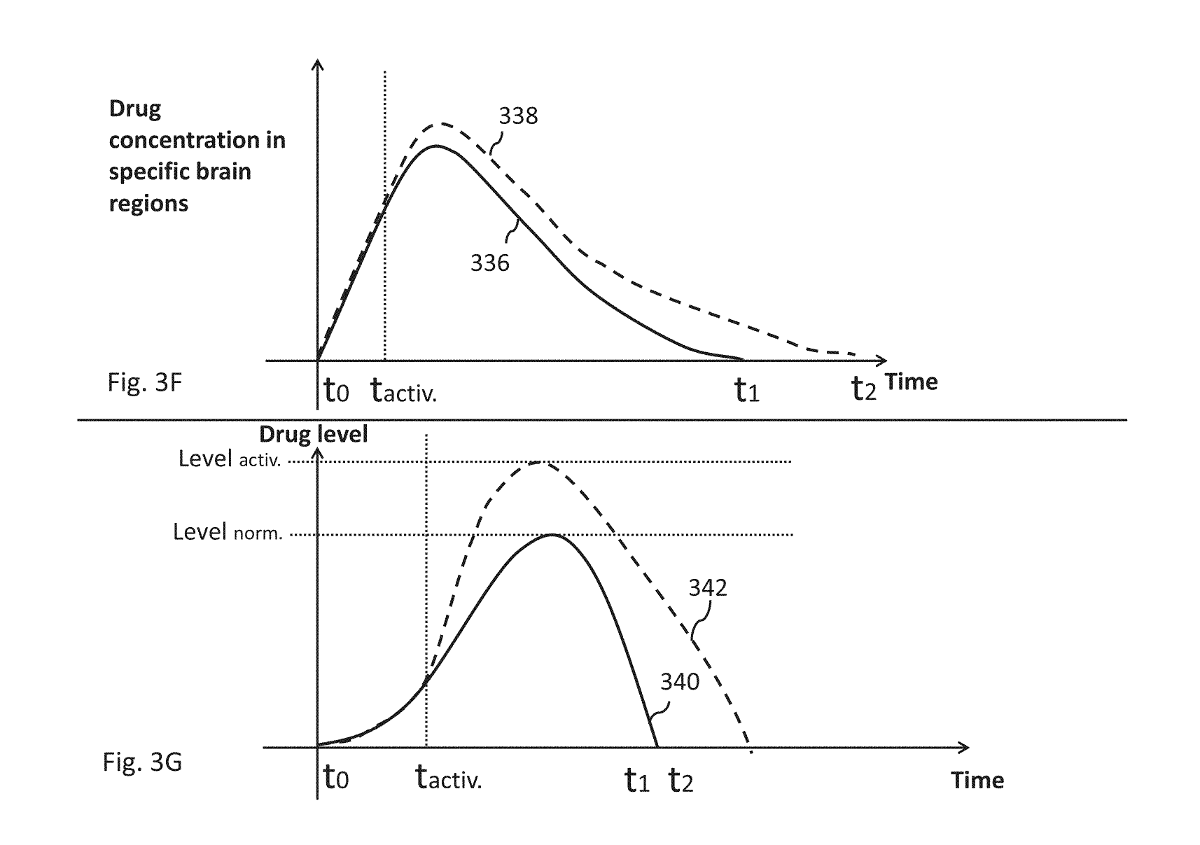

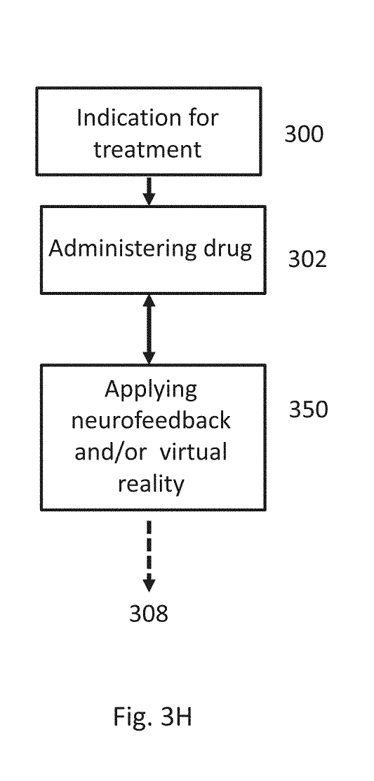

Reference is now being made to FIG. 3A depicting a process for application of an activation protocol, according to some embodiments of the invention.

According to some exemplary embodiments, a patient receives an indication for a treatment at 300. In some embodiments, the indication comprises the time schedule for the treatment, for example the timing parameters for drug administration and/or for the application of an activation protocol.

According to some exemplary embodiments, the drug is administered at 302. In some embodiments, the patient receives instructions regarding the activation protocol, for example, after the drug is administered. Alternatively, the patient receives the instructions before administering the drug. In some embodiments, the instructions and/or indications are delivered to the patient using a computer or a handheld device. In some embodiments, the instructions and/or indications are delivered by an application or a program installed on the computer or on the handheld device. In some embodiments, the instructions comprise the desired time interval between the drug administration and the activation protocol application. In some embodiments, the instructions comprise at least one desired position for placing an electrode or a device configured to deliver the activation treatment. Additionally or optionally, the instructions comprise at least one desired position and/or instructions for placing at least one sensor, for example to monitor at least one clinical parameter related to the treatment.

According to some exemplary embodiments, the drug is a breathable drug. In some embodiments, the drug is administered by inhalation.

According to some exemplary embodiments, the activation protocol is applied at 306. In some embodiments, the activation protocol is applied using a program and or an application stored in a computer or a handheld device. In some embodiments, the patient receives an indication for starting the activation protocol and/or an indication for ending the activation protocol.

Optionally, the effect of the treatment is determined at 308. In some embodiments, the effect of the treatment is determined, for example, by measuring EEG parameters. Alternatively or additionally, the effect of the treatment is determined by measuring at least one clinical parameter. In some embodiments, the effect of the treatment is determined by receiving a feedback from the patient, for example, the feeling of the patient after the treatment session. In some embodiments, the at least one clinical parameter and/or the EEG parameters are measured at least 10 minutes following the treatment, for example 60 minutes following the treatment. In some embodiments, the effect is tested via at least one psychometric and/or physical test. In some embodiments, if the effect of the drug is not a desired effect, then an indication is delivered to the patient and/or to an expert, for example a physician. In some embodiments, a modified activation treatment is delivered to the patient by the expert in response to the indication. Alternatively, an application or a program selects a modified activation protocol or a modified treatment protocol, in response to the indication.

According to some embodiments, at least one additional activation protocol is applied after the application of the first activation protocol at 306. In some embodiments, the additional activation protocol is applied, for example to prolong the effect of the first activation protocol. In some embodiments, the time difference between the application of the first activation protocol and the additional activation protocol is determined based, for example, on the pharmacokinetics and/or the pharmacodynamics of the drug.

### Exemplary Treatment Pharmacokinetics and Pharmacodynamics

Reference is now made to FIG. 3B depicting the change in drug concentration in specific brain regions over time, when an activation protocol is applied after administration of a drug, according to some embodiments of the invention. According to some exemplary embodiments, a drug is administered at to and an activation protocol is applied at t_{activ}., for example 1 minute, 5 minutes, 10 minutes, 15 minutes, 20 minutes, 25 minutes, or 30 minutes after drug administration. Alternatively, an activation protocol is applied before drug administration, for example 1 minute, 5 minutes, 10 minutes, 15 minutes, 20 minutes, 25 minutes, or 30 minutes after drug administration.

According to some exemplary embodiments, when an activation protocol is applied after drug administration, for example as described by curve 312, the drug concentration in the brain reaches a higher value over time, for example Conc.activ(concentration activation), compared to Conc.norm (Concentration normal) where an activation protocol is not applied, for example as described by curve 310. Alternatively, or additionally the drug remains for a longer period of time in the brain when an activation protocol is applied, for example as demonstrated by the value of t₂ of curve 312 which is larger compared to t₁ value of curve 310.

In some embodiments, when an activation protocol is applied at t_{activ}, the maximal concentration of the drug in at least one brain region is reached after a shorter period of time, compared to a treatment protocol where an activation protocol is not applied.

According to some exemplary embodiments, the amount of drug present in the brain, after the application of an activation protocol at is t_{activ}, higher compared to a treatment protocol where an activation protocol is not applied. In some embodiments, the amount of drug in the brain can be calculated, for example, by measuring the area under curve 312 to the area under curve 310.

According to some exemplary embodiments, application of an activation protocol increases the activity of selected brain regions, which leads to an increase in blood flow carrying drug molecules into the brain. In some embodiments, more drug molecules enter the brain following the activation protocol. In some embodiments, activation of selected brain regions, following administration of a drug, for example a psychotropic drug, leads to a reduced clearance rate of drug molecules from the brain.

Optionally, application of an activation protocol increases the drug's effect, for example by changing the reactivity of membrane receptors and/or transporters in the neuron. In some embodiments, application of an activation protocol increases the drug's effect, for example by changing association parameters between the drug molecules and active transporters molecules. Optionally, application of an activation protocol increases the association rate between the transporter molecules and the drug molecules.

Alternatively or additionally, the activation protocol increases the availability of transporter molecules in activated brain regions. In some embodiments, application of an activation protocol increases the penetration rate of transporter molecules and/or the penetration rate of drug-transporter complexes through the BBB and into the brain.

Reference is now made to FIG. 3C depicting the change in the drug effect over time, when an activation protocol is applied after administration of a drug, according to some embodiments of the invention.

According to some exemplary embodiments, a drug is administered at to, and an activation protocol is applied at t_{activ}, for example as previously described. In some embodiments, when an activation protocol is applied after drug administration at t_{activ}, for example, as depicted by curve 324, the maximal effect of the drug (Effect max) is reached earlier compared to a treatment protocol where an activation protocol is not applied, for example, as depicted by curve 322. In some embodiments, activation of selected brain regions, leads to accumulation of drug molecules in the activated brain regions. In some embodiments, the accumulation of the drug molecules leads, for example, to a faster response of the neural tissue to the drug.

Alternatively or additionally, the effect of the drug is prolonged when an activation protocol is applied. Optionally or additionally, the overall effect of the drug after application of an activation protocol is larger compared to the overall effect of the drug when an activation protocol is not applied, as demonstrated, for example, by the area under curve 324 which is larger compared to the area under curve 322.

Optionally or additionally, the maximal effect of the drug, when an activation protocol is applied is higher compared to a treatment protocol where the drug is administered but an activation protocol is not applied.

Reference is now made to FIG. 3D depicting the change in blood drug concentration over time when an activation protocol is applied after administration of a drug, according to some embodiments of the invention.

According to some exemplary embodiments, a drug is administered at t₀, followed by application of an activation protocol at t_{activ}, for example, as previously described. In some embodiments, when an activation protocol is applied after drug administration at t_{activ}, for example, as depicted by curve 328, the maximal blood drug concentration is achieved at an earlier time point compared to a treatment protocol where an activation protocol is not applied, for example, as depicted by curve 326. This effect is caused, in some embodiments, by the increase of drug delivery into the brain following the activation protocol.

According to some exemplary embodiments, the clearance of the drug from the blood is slower when an activation protocol is applied, compared to a treatment protocol when an activation protocol is not applied, for example as depicted by the larger value of t₂ compared to the value of t₁. Optionally, application of an activation protocol affects the clearance rate of unwanted compounds from the tissue.

Reference is now made to FIG. 3E depicting the change in the drug effect over time when applying an activation protocol following administration of a lower drug dosage, according to some embodiments of the invention.

According to some exemplary embodiments, when a drug, for example Ritalin® or Levodopa is administered at to, as depicted by graph 330 a desired effect is reached. In some embodiments, when administering a lower dosage of the drug, for example 50% of the dosage, a reduced drug effect is reached, as depicted by graph 332. In some embodiments, a reduced dosage of the drug is administered, for example to lower at least one side effect of the drug. In some embodiments, a lower drug dosage is administered at to, followed by application of an activation protocol at t_{activ}, as depicted by graph 334. Alternatively, an activation protocol is applied before the administration of a lower dosage of the drug. In some embodiments when an activation protocol is applied before or after the administration of a lower drug dose, the effect of the drug is higher compared to administration of the drug without application of the activation protocol. Optionally, the effect of a lower drug dose, for example 50% of the drug dose when an activation protocol is applied, is similar to the effect when administering 100% of the drug without activation protocol application.

Reference is now made to FIG. 3F depicting the change in the drug concentration in specific brain regions over time after an activation protocol is applied following the administration of a lower drug dosage, according to some embodiments of the invention.

According to some exemplary embodiments, when a drug for example Ritalin® or Levodopa is administered, it penetrates through the blood brain barrier (BBB), and enters the neural tissue, as depicted by graph 336. In some embodiments, when an activation protocol is applied after the drug administration, there is an increase in drug concentration in the neural tissue. In some embodiments, more drug molecules penetrate through the BBB and into the neural tissue following the activation protocol.

Optionally, there is an increase in drug concentration in at least one neural tissue region and/or reduction in drug concentration in other one or more neural tissue regions.

Reference is now made to FIG. 3G depicting the change in drug levels over time in the blood compartment of the brain following application of an activation protocol, according to some embodiments of the invention.

According to some exemplary embodiments, when a drug, for example Ritalin® or Levodopa or Haloperidol is administered, it is delivered through the blood flow into the blood compartment of the brain, before it penetrates through the BBB into the neural tissue as depicted by graph 340. In some embodiments, application of an activation protocol leads to an increase in blood flow and blood-borne drug molecules into the blood compartment of the brain, for example, as depicted by graph 342. Optionally, application of an activation protocol before or after drug administration leads to an increase in blood flow and in blood-borne drug molecules levels in at least one selected blood compartment region, for example, a blood compartment region near at least one activated brain region. Alternatively or additionally, application of an activation protocol before or after drug administration leads to a decrease in blood flow to at least one selected blood compartment region, for example blood compartment region near at least one brain regions with reduced activation levels. In some embodiments, the blood compartment of the brain comprises blood vessels that interact with neural tissue in the brain.

Exemplary Process for Application of an Activation Protocol Using Neurofeedback and/or Virtual Reality Techniques

Reference is now being made to FIG. 3H depicting a process for application of an activation protocol using virtual reality techniques, according to some embodiments of the invention.

According to some exemplary embodiments, a patient receives an indication for a treatment at 300, for example as previously shown in FIG. 3A. In some embodiments, a drug is administered at 302, for example as previously shown in FIG. 3A.

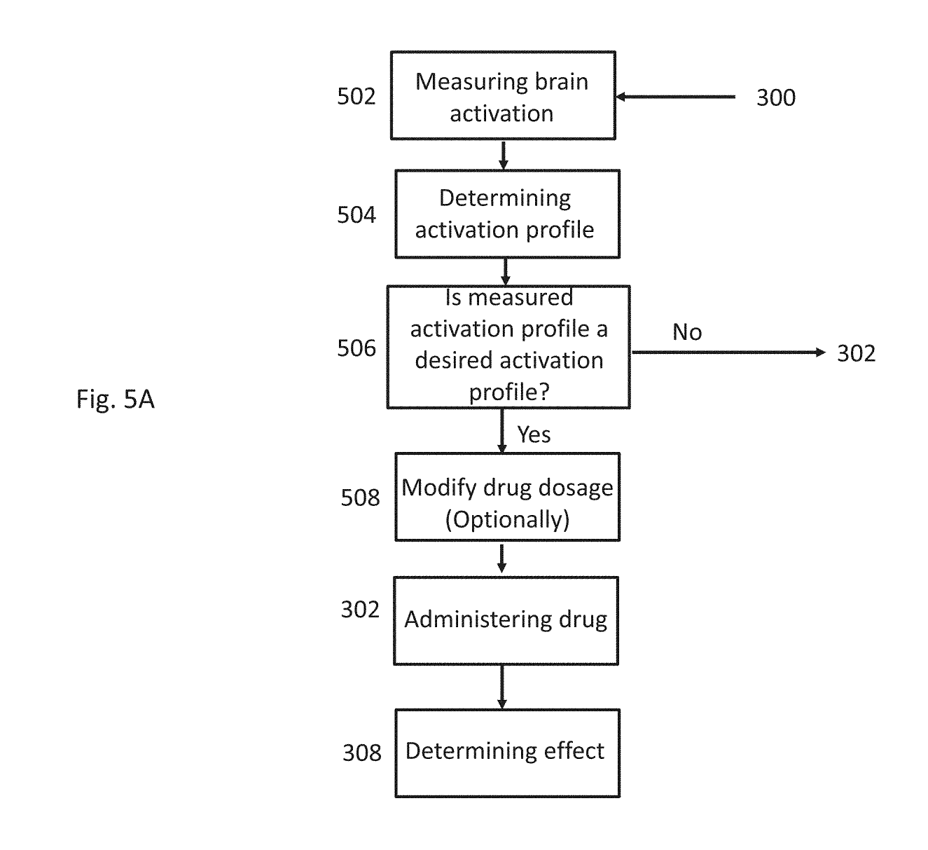

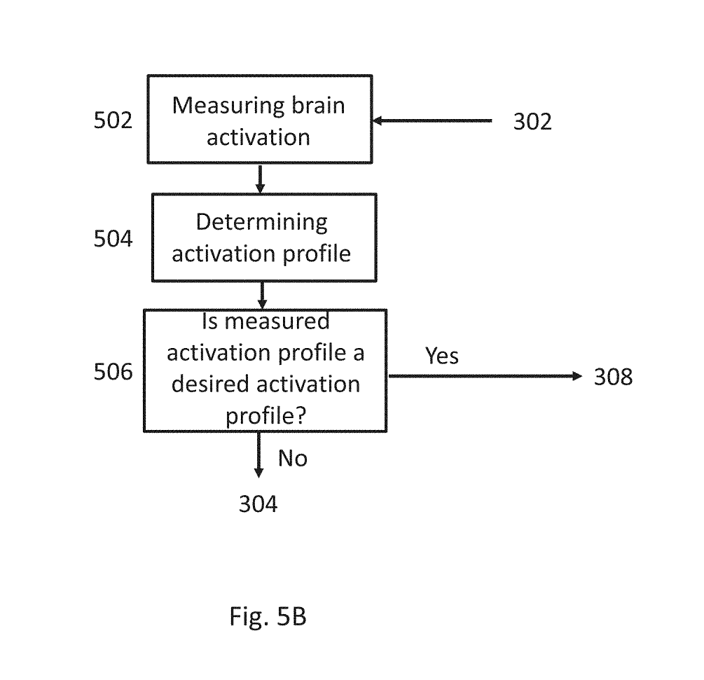

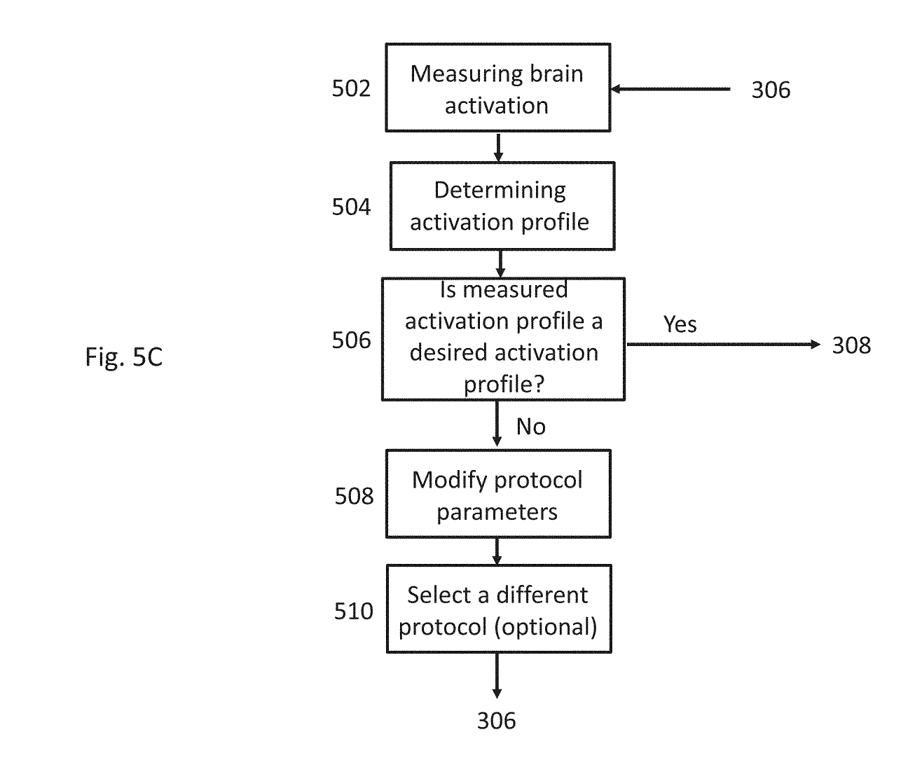

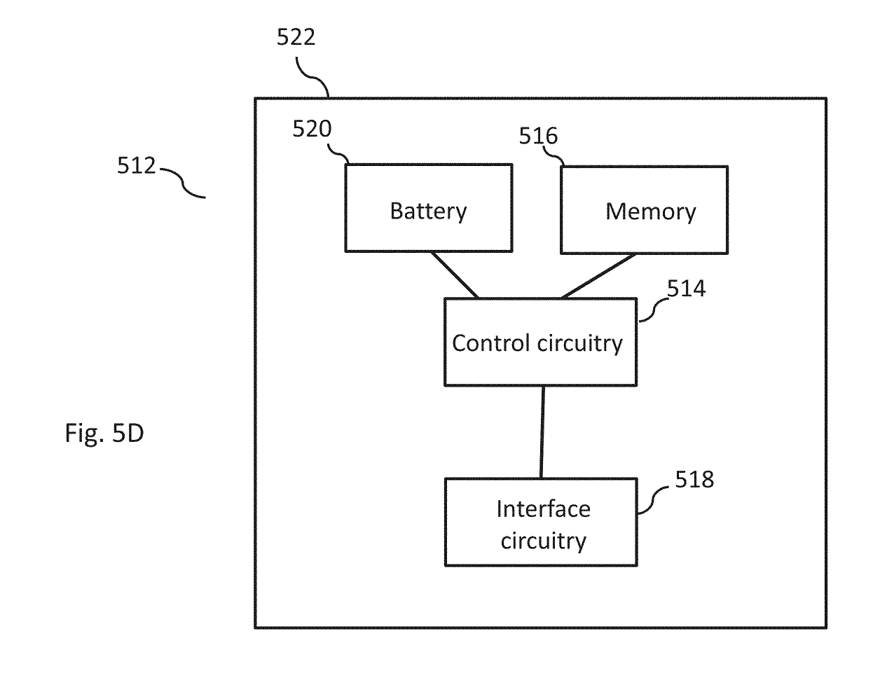

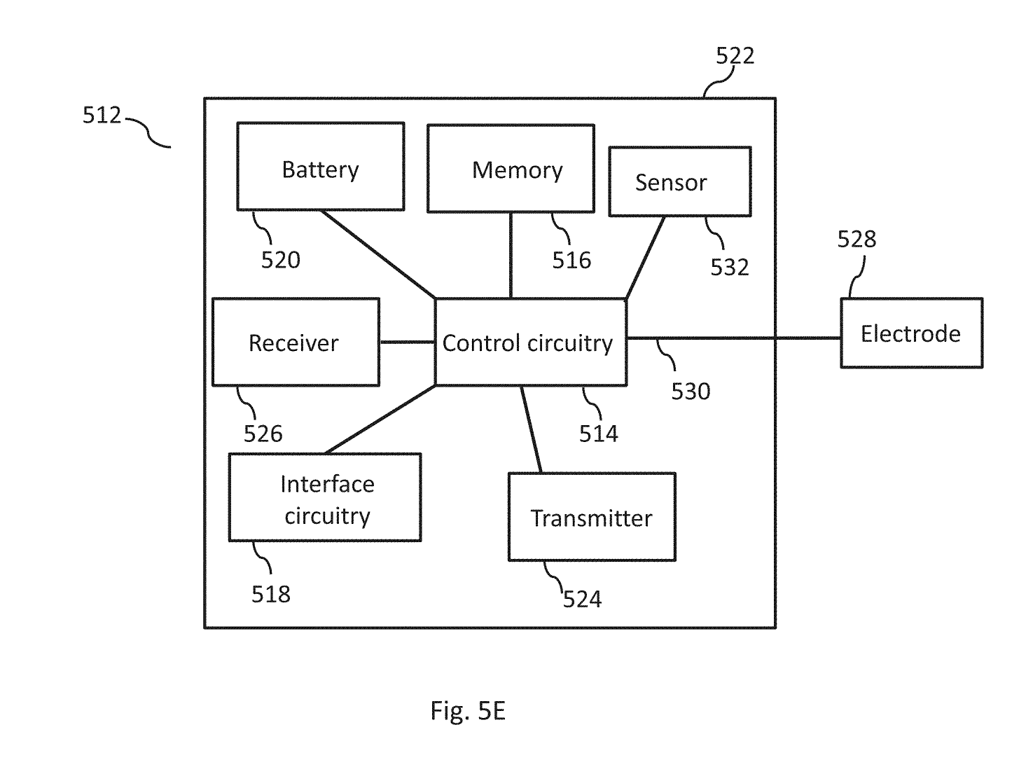

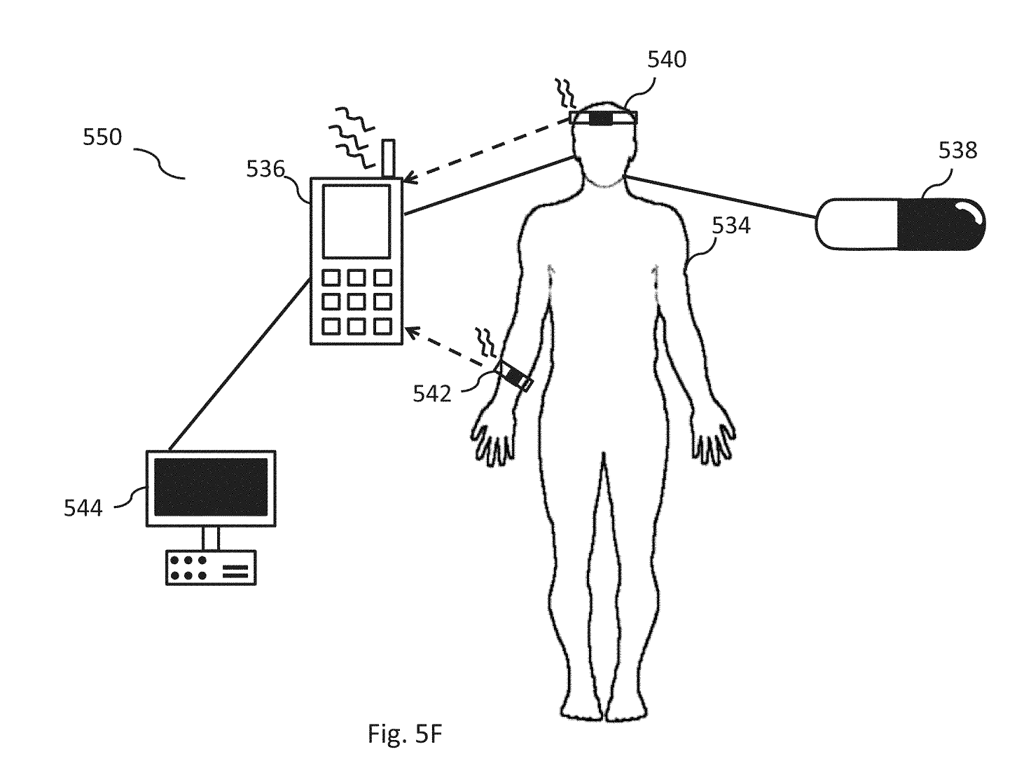

According to some exemplary embodiments, a neurofeedback protocol is applied at 350 optionally combined with a virtual reality protocol, for example to activate at least one brain region. In some embodiments, a virtual reality protocol is applied without a neurofeedback protocol. In some embodiments, the virtual reality protocol comprises recorded and/or presented scenarios, optionally in three-dimensions (3D). In some embodiments, the neurofeedback protocol and/or the virtual reality protocol/scenarios are delivered to a subject or a patient via a mobile device, for example a cellular device or a mobile computer, for example handheld device 536 shown in FIG. 5F.

In some embodiments, when treating a patient suffering from a type of phobia, for example fear of height and/or agoraphobia and/or snakes, an SSRI or an anxiolytic drug is administered, for example at 302. In some embodiments, a neurofeedback protocol is applied before, during or after the administration of the drug, for example to down-regulate the activity of the amygdala and/or to up regulate the activity of the IFG. In some embodiments, the neurofeedback protocol is applied while interfacing with at least one virtual reality scenario, for example at least one virtual reality scenario which corresponds to the phobic object or environment.

In some embodiments, when treating a patient suffering from PTSD, for example suffering from arousal and/or re-experience, an anxiolytic or an anticonvulsant drug is administered, for example at 302 In some embodiments, a neurofeedback protocol is applied before, during or after the administration of the drug, for example to regulate the activity of the amygdala and/or hippocampus and/or locus ceruleus. In some embodiments, the neurofeedback protocol is applied while interfacing with at least one virtual reality scenario, for example at least one virtual reality scenario related to a traumatic story. In some embodiments, which corresponds to the phobic object or environment.

In some embodiments, when treating a patient suffering from PTSD, for example suffering from avoidance, a Bupropion drug is administered, for example at 302. In some embodiments, In some embodiments, a neurofeedback protocol is applied before, during or after the administration of the drug, for example to up-regulate the activity of the dorsal ACCC/preSMA. In some embodiments, the neurofeedback protocol is applied while interfacing with at least one virtual reality scenario, for example at least one virtual reality scenario demonstrating goal-directed situations that demand motivation decision making.

In some embodiments, when treating a patient suffering from ADHD symptoms, for example suffering from attention deficits and/or impulsivity, Ritalin ® drug is administered, for example at 302. In some embodiments, a neurofeedback protocol is applied before, during or after the administration of the drug, for example to up-regulate the activity of the right-IFG. In some embodiments, the neurofeedback protocol is applied while interfacing with at least one virtual reality scenario, for example at least one virtual reality scenario where the patient is required to perform (or not to perform) actions according to cues appearing in high or low probabilities. In some embodiments, the virtual reality scenarios are applied without neurofeedback.

In some embodiments, when treating a patient suffering from chronic pain, at least one analgesic drug is administered, for example at 302. In some embodiments, a neurofeedback protocol is applied before, during or after the administration of the drug, for example to up-regulate the activity of the viscero-somatic system (including insula cortex, somatosensory cortex and cingulate cortex. In some embodiments, the neurofeedback protocol is applied while interfacing with at least one virtual reality scenario, for example at least one virtual reality scenario optionally demonstrating the presence near or in a room in-fire, including various levels of heat sensations.

In some embodiments, when treating a patient suffering from social anxiety, an SSRI drug is administered, for example at 302. In some embodiments, a neurofeedback protocol is applied before, during or after the administration of the drug, for example to up-regulate the activity of at least one brain region related to self-referential, for example the vmPFC region. In some embodiments, the neurofeedback protocol is applied while interfacing with at least one virtual reality scenario, for example at least one virtual reality scenario demonstrating at least one person socially interacting with the patient, for example speaking with the patient from varying distances.

In some embodiments, when treating a patient suffering from depressive mood symptoms, an SSRI drug is administered, for example at 302. In some embodiments, a neurofeedback protocol is applied before, during or after the administration of the drug, for example to up-regulate the activity of at least one brain region related to the reward system, for example the nucleaus accumbens and/or vmPFC brain regions. In some embodiments, the neurofeedback protocol is applied while interfacing with at least one virtual reality scenario, for example at least one virtual reality scenario demonstrating a rewarding cue, for example a highly desired rewarding cue in a context of a game, for example the hidden money game.

### Exemplary Process for Selection of an Activation Treatment

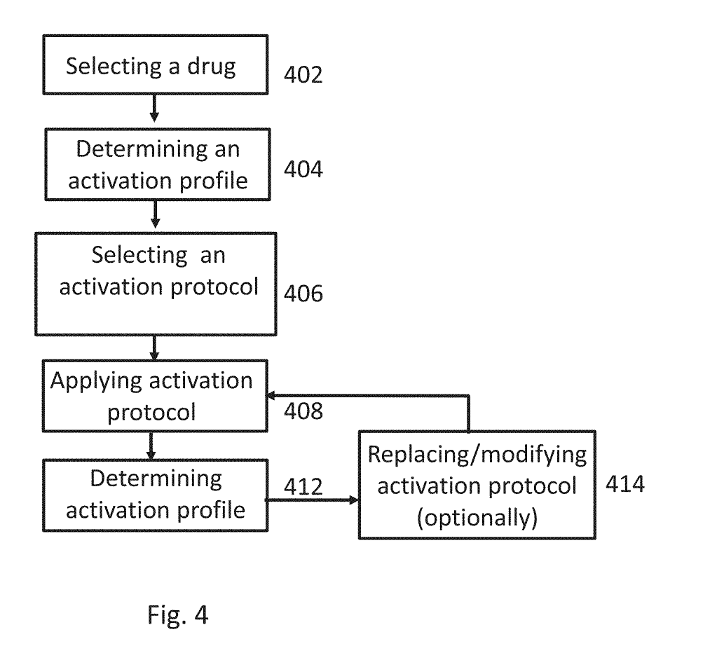

Reference is now made to FIG. 4 depicting a process for selecting an activation treatment, according to some embodiments of the invention.

According to some exemplary embodiments, a patient, for example a patient suffering from at least one neurological and/or psychiatric symptom is diagnosed using cognitive assays. Optionally, the patient is diagnosed using at least one imaging technique, for example CT, MRI, fMRI, or ultrasound.

According to some exemplary embodiments, at least one drug is selected at 402 for the treatment of the at least one neurological and/or psychiatric symptom of the patient. In some embodiments, the drug is selected to treat the clinical condition of the patient.

According to some exemplary embodiments, the desired activation profile of the selected drug is determined at 404. In some embodiments, the activation profile comprises at least one desired brain region and/or neuronal network to be affected by the selected drug. Optionally, the activation profile of the selected drug comprises at least one undesired brain region and/or neuronal network to be affected by the selected drug.

According to some exemplary embodiments, an activation treatment, for example a cognitive exercise, a neurofeedback treatment or a TMS treatment, is selected at 406, for example to match the desired activation profile of the drug. In some embodiments, the activation treatment is selected from a plurality of activation treatments that selectively match, for example, a desired activation profile and/or a drug.

In some embodiments, at least one treatment protocol parameter is adjusted to match a desired activation profile, for example as determined at 404.

According to some exemplary embodiments, the activation protocol is applied at 408. In some embodiments, the activation profile of at least one brain region and/or at least one neuronal network is determined at 412, for example by measuring EEG parameters or by monitoring at least one clinical parameter. Optionally, the activation profile is determined using, an imaging technique, for example fMRI.

In some embodiments, the activation profile is determined during the application of the activation protocol. Alternatively or additionally, the activation profile is determined at least 10 minutes following the activation treatment, for example 10 minutes, 15 minutes, 20 minutes, 30 minutes or 1 hour. In some embodiments, the time period required for determination of the application profile depends on pharmacodynamics and/or pharmacokinetics properties of the drug.

In some embodiments, the activation protocol or activation protocols are modified according to the measured activation profile. Optionally, the activation protocol intensity and/or duration and/or type of cognitive and/or physical task are modified according to the measured activation profile.

Alternatively or additionally, the drug dosage is modified according to the measured activation profile. In some embodiments, if the measured activation profile is not the desired activation profile then the drug dosage is increased and/or the dug administration protocol is modified. In some embodiments, if the measured activation profile is the desired activation protocol then the drug dosage is lowered, for example during a calibration process of the combined drug-activation protocol treatment.

In some embodiments, if the desired activation profile is reached then an indication is delivered to the patient. In some embodiments, if the desired activation profile is not reached, then the activation protocol is modified or replaced at 414. In some embodiments, if the desired activation profile is not reached at least one parameter of the activation protocol is modified, for example the duration of the treatment.

According to some embodiments, the activation protocol is modified or replaced to reach desired activation levels of at least one specific brain region and/or to reach desired connectivity measures of at least one neuronal network. Optionally, the desired activation levels and/or the desired connectivity measures are determined optionally in a clinic by an expert, for example a neurologist. In some embodments, the desired activation levels and/or the desired connectivity are determined using EEG, MRI, functional MRI, EMG or any other method suitable for monitoring brain activity and/or the effect of brain activity on other parts of the body, for example the muscles.

According to some embodiments, the application protocol is modified using explicit neurofeedback or covert neurofeedback methods. Covert neurofeedback refers to a neurofeedback protocol in which the subject is not asked to modulate her brain signals volitionally. In covert neurofeedback, the subject's brain reactions to specific external stimuli are probed and these stimuli are modified to elicit desired brain states without the subject's awareness. In some embodiments, for example, when presenting a set of stimuli to the subject in order to trigger local brain activation, the most effective stimulus may be selected and reused to optimize the activation protocol. Alternatively or additionally, in a different scenario, spontaneous activation of a specific region may be reinforced by rewarding cues, while the subject is unaware of the cause of the reward.

According to some exemplary embodiments, the new or the modified activation protocol is delivered to the patient, for example, to a handheld device of the patient. In some embodiments, the new or modified activation protocol is applied at 308 and the activation profile is determined as previously described.

### Exemplary Process for Application of an Activation Protocol

Reference is now made to FIGS. 5A-5C, depicting a process for application of an activation treatment, according to some embodiments of the invention.

According to some exemplary embodiments, after an indication for a treatment is delivered at 300, the brain activation profile is measured at 502. In some embodiments, the brain activation profile is measured by at least two electrodes connected to the head of the patient. Alternatively, the brain activation profile is determined using a cognitive task.

In some embodiments, the activation profile of the brain is measured before the administration of a drug or an application of an activation protocol to determine, for example, whether the activation profile of the brain is a desired activation profile and therefore there is no need to apply the activation protocol. Additionally, if the application profile is a desired application profile then, optionally, the drug dosage can be modified.

According to some exemplary embodiments, the activation profile is determined at 504. In some embodiments, the determined activation profile is compared to a desired activation profile at 506. In some embodiments, if the measured activation profile is not a desired activation profile then an indication is delivered to the patient and/or to an expert for example a physician. Additionally, a drug is administered at 302. In some embodiments, the indication is provided to the user as part of a game-like activation protocol.

According to some exemplary embodiments, if the measured activation profile is a desired activation profile then an activation protocol is not applied. Optionally, the drug dosage is modified based on the activation profile parameters, for example the drug dosage is reduced by 10%, 25% or 50%.

According to some exemplary embodiments, a drug is administered at 302 and the treatment effect is determined, for example as previously described.

According to some exemplary embodiments, as described in FIG. 5B, after the administration of a drug at 302, brain activation is measured at 502, and the activation profile is determined at 504 as previously described.

In some embodiments, the measured activation profile is compared to a desired application profile at 506. In some embodiments, if the measured activation is a desired activation profile then an activation protocol is not applied, and the treatment effect is determined at 308, for example as preciously described.

Alternatively, if the measured activation profile is not a desired activation profile, then an activation profile is applied at 304, for example as described in FIG. 5A.

According to some exemplary embodiments, as described in FIG. 5C, after the application of an activation protocol at 306, brain activation is measured at 502 and an activation profile is determined, for example as previously described. In some embodiments, the measured activation profile is compared to a desired activation profile at 506, to decide whether the measured activation profile is a desired application profile. In some embodiments, if the measured activation profile is a desired activation profile, then the treatment effect is determined at 308, as previously described.

In some embodiments, if the measured activation profile is not a desired activation profile, then at least one parameter of the activation protocol is modified at 508. Alternatively, a different activation protocol is selected at 510. Optionally, an indication is delivered to the patient, and to an expert, for example a physician if the measured activation profile is not a desired activation profile.

In some embodiments, the different activation protocol or the modified activation protocol is applied at 306, for example, as described at FIG. 3A.

### Exemplary Device for Application of a Brain Activation Protocol

Reference is now made to FIGS. 5D-5E, depicting a device for application of a brain activation protocol, according to some embodiments of the invention.

According to some exemplary embodiments, device 512 comprises a memory 516 for storing at least one treatment protocol and/or at least one activation protocol. In some embodiments, memory 516 stores log files of device 512.

In some embodiments, a control circuitry 514 delivers instructions and/or at least one indication to a patient and/or an expert via an interface circuitry 518. In some embodiments, control circuitry 514 reads and/or writes log files and/or at least one treatment protocol to memory 516. Additionally, control circuitry 514 reads and/or writes at least one activation protocol to memory 516.

In some embodiments, device 512 comprises a battery 520, connected to control circuitry 514. In some embodiments, battery 520 is a rechargeable battery, for example a lithium-ion battery. In some embodiments, if battery 520 is at least 50% discharged, for example 75%, 85%, 90%, 95% then control circuitry 514 delivers an indication, for example an alert signal to the patient via interface circuitry 518. In some embodiments, a patient and/or an expert and/or a caregiver delivers feedback parameters via interface circuitry 518.

In some embodiments, device comprises a casing 522. In some embodiments, casing 522 comprises at least one attachment and detachment member configured to attach device 512 to the body or to the clothes of the patient.

According to some exemplary embodiments, device 512 comprises at least one electrode connected to the body of the patient. In some embodiments, the at least one electrode measures at least one clinical parameter of the body, for example EEG parameters. In some embodiments, electrode 528 delivers the measured parameters to control circuitry 514 through wire 530.

According to some exemplary embodiments, device 512 comprises at least one sensor 532 for measuring at least one clinical parameter, for example skin conductance, heart rate, blood pressure, blood flow parameters or pupil diameter. In some embodiments, sensor 532 and/or electrode 528 measures at least one clinical parameter before and/or after the administration of a drug, for example to determine the activation profile of the brain. Optionally or additionally, sensor 532 and/or electrode 528 measure at least one clinical parameter, for example to determine the treatment effect on the patient. In some embodiments, the parameters measured by sensor 532 and/or electrode 528 are stored in memory 532.

According to some exemplary embodiments, in response to the measured clinical parameter and/or to the received feedback, control circuitry 514 modifies the treatment protocol and/or the activation protocol. Optionally, in response to the measured clinical parameter and/or to the received feedback, control circuitry 514 stops the treatment protocol and/or the activation protocol. In some embodiments, in response to the measured clinical parameter and/or to the received feedback, control circuitry 514 changes the dosage of the drug and/or the administration procedure, for example administration time schedule.

According to some exemplary embodiments, device 512 delivers an indication to a remote computer and/or a handheld device via transmitter 524, for example when the measured activity profile is not a desired profile or when the determined treatment effect is not a desired treatment effect. In some embodiments, the information transferred via transmitter 524 includes parameters stored in memory 516, for example feedback parameters received from the patient via interface circuitry 518.

According to some exemplary embodiments, device 512 receives information from a remote computer and/or a handheld device via receiver 526. In some embodiments, the information delivered through receiver 526 comprises instructions to the patient, and/or modifications of an activation protocol and/or a new activation protocol. In some embodiments, the information received by receiver 526 is stored in memory 526.

### Exemplary System for Application of an Activation Protocol

Reference is now made to FIG. 5F depicting a system for application of an activation protocol, according to some embodiments of the invention.

According to some exemplary embodiments, system 550 comprises a handheld device 526 for example, for delivering at least one indication to patient 534. In some embodiments, handheld device 536 delivers instructions regarding the treatment protocol and/or regarding the activation protocol to patient 534 before, during or after the administration of drug 538.

According to some exemplary embodiments, system 550 comprises at least one sensor, for example sensor 542 or electrode for measuring at least one clinical parameter of patient 534, for example EEG parameters, heart rate parameters, blood pressure parameters, skin conductance parameters, blood flow parameters or pupil diameter. In some embodiments, sensor 542 and/or electrode 540 delivers the measured parameter to handheld device 536 via a wireless signal. Optionally, the signal is delivered from sensor 542 or electrode 540 via a wire. In some embodiments, sensor 542 and/or electrode 540 measures the at least one clinical parameter before, during, or after the administration of drug 538.

According to some exemplary embodiments, a remote computer 544 delivers instructions and/or at least one treatment protocol and/or at least one activation protocol to handheld device 536. In some embodiments, handheld device 536 delivers an indication to remote computer 544, for example when the measured clinical parameter is not in a desired range of parameters. Optionally, patient 534 delivers feedback to handheld device 536. In some embodiments, handheld device 536 transmits the feedback to remote computer 544.

### Exemplary Application of an Activation Protocol Using a Handheld Device

According to some exemplary embodiments, a patient, for example patient 534 receives a human detectable indication from a handheld device, for example handheld device 536 before the administration of drug 538. In some embodiments, the indication is delivered according to a treatment protocol stored in the memory of handheld device 536.

In some embodiments, after the administration of drug 538, patient 534 receives instructions for application of an activation protocol, by a user interface of handheld device 536. In some embodiments, the instructions comprise instructions for application of at least one cognitive task and/or at least one physical task. Additionally or optionally, the instructions comprise the duration of each task and/or the number of repetitions for each task and the time interval between drug administration and application of an activation protocol. In some embodiments, the instructions comprise the time interval between each task and the following task.

In some embodiments, when the activation protocol is applied a human detectable indication is delivered by the handheld device to the patient. In some embodiments, the indication comprise the time to the next drug administration and/or to the next application of an activation protocol.

In some embodiments, the handheld device measures at least one cognitive and/or clinical parameter before and/or after the application of an activation protocol.

Alternatively or additionally, the handheld device measures at least one cognitive and/or clinical parameter before and/or after the administration of the drug. In some embodiments, the at least one cognitive and/or clinical parameter is measured by at least one sensor or electrode connected to the body of the patient, for example to the head and/or to the hand and/or to the chest of the patient. In some embodiments, the at least one sensor transmits the at least one cognitive and/or clinical parameter to the handheld device by a wireless signal. Alternatively, the at least one sensor or electrode transmits the at least one cognitive and/or clinical parameter via a wire connected to the handheld device.

In some embodiments, a program on the handheld device determines whether the measured cognitive and/or clinical parameter value is a desired clinical and/or cognitive value. In some embodiments, if the measured cognitive and/or clinical parameter value is a desired value, then handheld device 536 delivers an indication to patient 534, for example a treatment compliance indication. Alternatively, if the measured parameter value is not a desired value, then handheld device 536 modifies the treatment protocol which comprises the drug administration and activation protocol application, for example the handheld device modifies drug dosage and/or the interval between drug administration and application of an activation protocol. Optionally, the handheld device modifies at least one parameter of the activation protocol, for example the duration of the activation protocol, the type of the cognitive and/or the physical task, the number of repetitions of the task.

### Exemplary Activation Mechanism of an Activation Protocol

According to some embodiments, a patient, for example patient 534, is administered with a Norepinephrine Reuptake Inhibitor, for example Venlafaxine.

Optionally, Venlafaxine is administered to treat Anxiety and/or at least one symptom related to Anxiety. In some embodiments, an activation protocol is applied 5-90 minutes after Venlafaxine administration, for example 5-40 minutes, 30-60 minutes or 50-90 minutes. In some embodiments, the activation protocol will be applied for 5-60 minutes, for example 10-30 minutes, 25-50 minutes or 30-45 minutes. In some embodiments, the activation protocol directs Venlafaxine at least partially to the Amygdala.

In some embodiments, the activation protocol comprises viewing of dynamic emotional content, for example movies, optionally by using virtual reality or augmented reality means. Alternatively or additionally, the activation protocol comprises listening to music. In some embodiments, the activation protocol will be applied using a handheld device, for example handheld device 536.

According to some embodiments, a patient, for example patient 534, is administered with MPH, for example to treat attention deficits and/or at least one symptom associated with attention deficits. In some embodiments, an activation protocol is applied 5-90 minutes after MPH administration, for example 5-40 minutes, 30-60 minutes or 50-90 minutes. In some embodiments, the activation protocol will be applied for 5-60 minutes, for example 10-30 minutes, 25-50 minutes or 30-45 minutes. In some embodiments, the activation protocol directs MPH, at least partially to dorsolateral and/or ventrolateral prefrontal brain regions.

In some embodiments, the activation protocol comprises at least one task related to executive function and/or control inhibition, for example go-no go and/or N-back tasks. In some embodiments, the activation protocol will be applied using a handheld device, for example handheld device 536.

According to some embodiments, a patient, for example patient 534, is administered with Bupropion, for example to treat post-traumatic stress disorder (PTSD) and/or at least one symptom associated with PTSD. In some embodiments, an activation protocol is applied 5-90 minutes after Bupropion administration, for example 5-40 minutes, 20-60 minutes or 50-90 minutes. In some embodiments, the activation protocol will be applied for 5-60 minutes, for example 10-30 minutes, 25-50 minutes or 30-45 minutes. In some embodiments, the activation protocol directs Bupropion at least partially to the dorsal anterior cingulate cortex brain region.

In some embodiments, the activation protocol comprises at least one Action planning related task for example, an object manipulation task, a grid sailing task which includes the moving of a cursor with at least one finger to a selected target position, and/or a finger tapping task which include instructed and non-instructed finger tapping sequences. In some embodiments, the activation protocol is applied using a handheld device, for example handheld device 536.

According to some embodiments, a patient, for example patient 534, is administered with Reboxetine, for example to treat MCI and/or at least one symptom associated with MCI. In some embodiments, an activation protocol is applied 5-90 minutes after Reboxetine administration, for example 5-40 minutes, 30-60 minutes or 50-90 minutes. In some embodiments, the activation protocol is applied for 5-60 minutes, for example 10-30 minutes, 25-50 minutes or 30-45 minutes. In some embodiments, the activation protocol directs the drug at least partially to the hippocampus.

In some embodiments, the activation protocol comprises at least one task related to memory, for example a paired association task which relates to verbal declarative memory, an N-back task or a Digital Span Test which relate to working memory, and/or a verbal generation task which relate to episodic memory. In some embodiments, the activation protocol is applied using a handheld device, for example handheld device 536.

According to some embodiments, a patient, for example patient 534, is administered with Levodopa, for example to treat Parkinson's disease. In some embodiments, an activation protocol is applied 5-90 minutes after Levodopa administration, for example 5-40 minutes, 30-60 minutes or 50-90 minutes. In some embodiments, the activation protocol will be applied for 5-60 minutes, for example 10-30 minutes, 25-50 minutes or 30-45 minutes. In some embodiments, the activation protocol directs Levodopa at least partially to the caudate nucleus brain region.

Optionally, the applied activation protocol directs Levodopa at least partially away from mesocortical brain regions.

In some embodiments, the activation protocol comprises at least one task related to motor actions, for example game-like scenarios. In some embodiments, the activation protocol is applied using a handheld device, for example handheld device 536.

Optionally, the motor action related task is applied using virtual reality or augmented reality means.

Exemplary Application of an Activation Protocol Combined with Inhalation of Hyperbaric Gas

According to some exemplary embodiments, an activation protocol is applied together with a hyperbaric gas, for example during a hyperbaric medicine treatment. In some embodiments, the activation protocol is applied before, during or after the inhalation of the hyperbaric gas. In some embodiments, unlike psychotropic drugs that are directed mostly to affect the brain or tissues of the nervous system, the hyperbaric gas acts systemically on the entire body and is not directed to the brain under normal conditions. Therefore, and without being bound by theory, application of an activation protocol increases the probability of the hyperbaric gas to enter the brain and to enable a more specific effect, optionally a more robust effect compared to normal conditions, on the activated brain regions.

In some embodiments, a treatment session combining application of an activation protocol in combination with inhalation of a hyperbaric gas lasts between 15-120 minutes, for example 30 minutes, 45 minutes, or 60 minutes. In some embodiments a treatment is repeated at least twice a week, for example 3 times, 4 times, 5 times or 6 times in a week. Alternatively, the treatment session is applied only once, for example as a treatment to an acute disease or condition. In some embodiments, the maximal pressure of the gas during the hyperbaric treatment is in the range of 1.5 bar to 8 bar. In some embodiments, the average concentration of the hyperbaric gas in the breathable air is between 25-100%, for example 25-60%, 50-75% or 70-100%.

In some embodiments, the combination of an activation protocol with a hyperbaric treatment is used to treat stroke, traumatic brain ischemia (TBI), fibromyalgia, Asperger syndrome and autism.

In some embodiments, for treating stroke and/or TBI an application protocol is applied to activate damaged brain regions. Alternatively, the application protocol is applied to increase the activation of undamaged brain regions, for example to compensate for the loss of the damaged regions.

In some embodiments, an application protocol is applied to activate at least one brain region related to autism including the superior temporal sulcus, the temporoparietal junction, the insula and the premotor cortex during hyperbaric treatment to induce neurogenesis in the target regions.

### Exemplary Validation Experiments

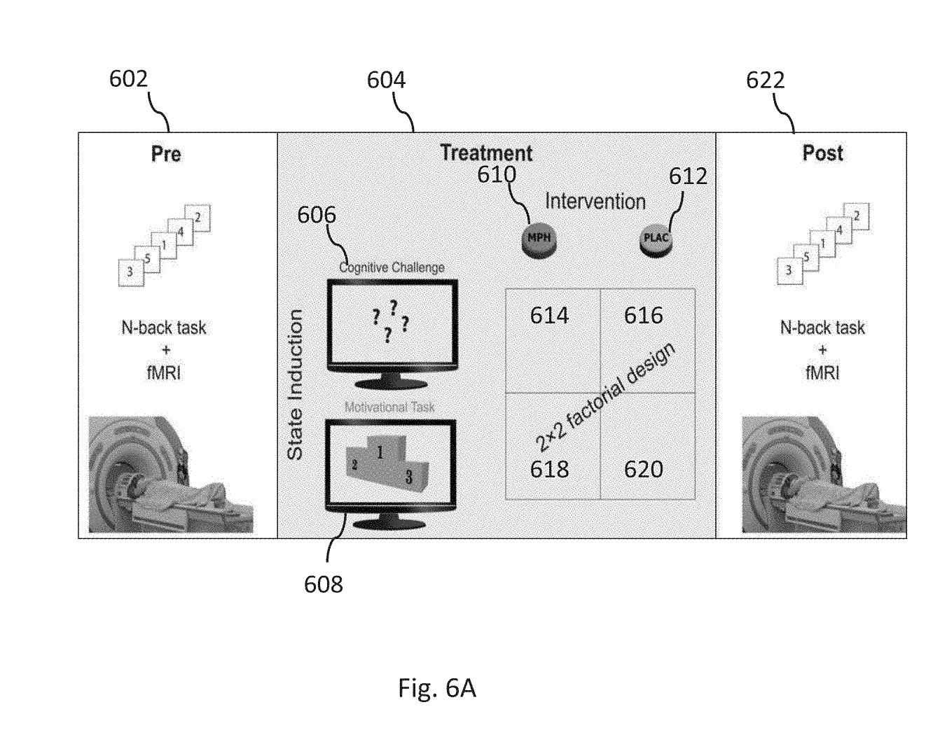

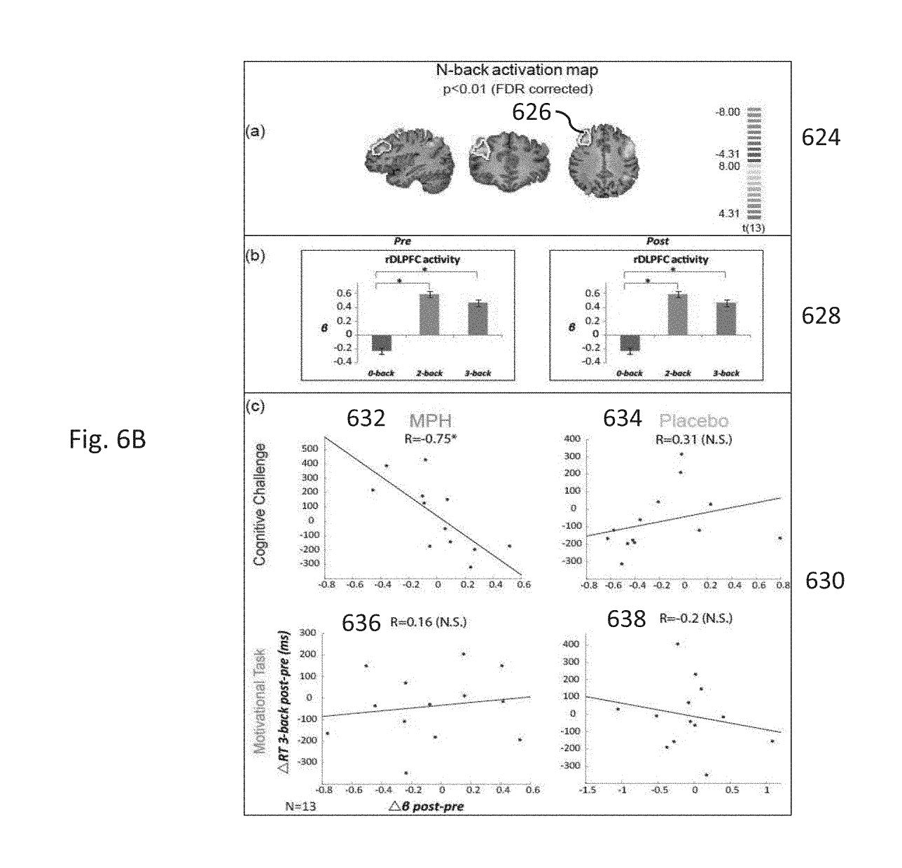

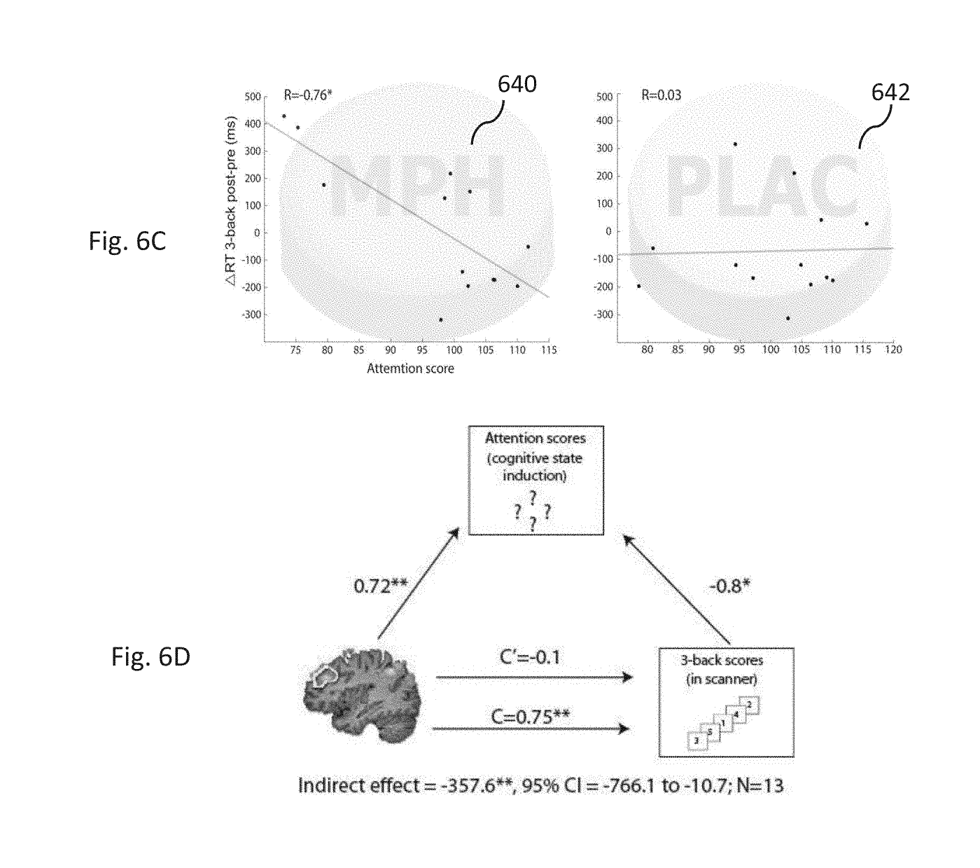

Reference is now made to FIGS. 6A-6D depicting a validation experiment comparing the coupling of an activation protocol with administration of Methylphenidate (MPH, Ritalin®) to coupling the activation protocol with placebo administration, according to some embodiments of the invention.

According to some exemplary embodiments, an N-back task is performed together with fMRI analysis at 602 before the application of an activation treatment and drug administration. In some embodiments, the N-back task measures the working memory. In some embodiments, performing fMRI while performing the N-back task allows, for example, to identify which brain region is involved when working memory is used.

According to some exemplary embodiments, during treatment 604, a cognitive challenge 606 was coupled with MPH administration 610 and compared to a motivational task 608 and administration of placebo (PLAC) 612, in a 2×2 factorial design. In some embodiments, the cognitive challenge 606 and the motivational task 608 are performed after the MPH 610 and the PLAC 612 administration. In some embodiments, group 614 received MPH 610 and performed cognitive challenge 606. In some embodiments, group 616 received PLAC 612 and performed cognitive challenge 606. In some embodiments, group 618 received MPH and performed a motivational task 608. In some embodiments, a motivational task comprises a task that involves expectation for reward and/or punishment. In some embodiments, group 620 received PLAC and performed a motivational task 608. In some embodiments, cognitive challenge was performed using the NeuroTrax computerized cognitive testing (NeuroTrax Corp., Bellaire, Tex.). In some embodiments, the NeuroTrax computerized cognitive testing is designed for assessing ADHD-related measures such as attention and visual-spatial performance (Auriel et al., 2006). In some embodiments, the NeuroTrax computerized cognitive testing comprises a battery of a GoNoGo, Stroop interference, and non-verbal memory tasks.

In some embodiments, groups 614, 616, 618 and 620 performed the N-back task 60-90 minutes after treatment 604, while performing fMRI, at 622.

According to some exemplary embodiments, when using fMRI analysis while performing the N-back task, a whole brain activation map is generated at 624. In some embodiments, the brain activation map demonstrates the activation of brain regions related to executive functional network, for example frontal, parietal, and parahippocampal regions. In some embodiments, the right dorsolateral prefrontal cortex (rDLPFC) 626 is activated.

According to some exemplary embodiments, the activation of the rDLPFC is examined using fMRI when performing a 0-back task (control), a 2-back task and a 3-back task before and after treatment 604. In some embodiments the rDLPFC region was shown to mediate N-back performance (Owen et al., 2005). In some embodiments, an increased activity of the rDLPFC is demonstrated when performing 2-back task and 3-back task compared to 0-back task, at 628.

According to some exemplary embodiments, the link between post-pre rDLPFC activity change and the corresponding change in N-back performance is examined in 630. In some embodiments, in the MPH/cognitive condition 632, the change in the post-pre dLPFC beta weights difference negatively correlated with the post-pre change in reaction time (R=−0.75, p=0.003). In some embodiments, this indicates that a greater increase in 3-back-related rDLPFC activation following MPH/cognitive induction corresponded to larger decreases in reaction time during scanning. Additionally, this effect was not found for the other induction conditions (MPH/motivation 636: R=0.15; p=0.61, PLAC/cognitive 634: R=0.3; p=0.3, PLAC/motivation 638: R=−0.19; p=0.52). In some embodiments, a Steiger's Z-test indicated a significant difference between the correlations and a unique dLPFC beta-RT association only when the MPH administration coincided with cognitive tasks (Steiger's Z=−2.934, p<0.05, bonferroni-corrected for multiple comparisons).

According to some exemplary embodiments, subjects who were more successful in recruiting their attention during the cognitive induction state benefited more from the functional-pharmacological coupling. In some embodiments, NeuroTrax attention scores negatively correlated with 3-back (maximal cognitive load) RT post-pre difference in the MPH 640 (R=−0.76, p<0.01). Additionally, no significant correlation was found in the placebo condition 642 (R=0.03, ns). In some embodiments, the lack of a significant correlation in the placebo condition 642 indicates, for example, that the improvement is not explained by general capabilities of specific participants, but is rather specific to the performance during the induction condition.

According to some exemplary embodiments, fMRI data that were collected during the performance of the N-back task indicate that the FPC is mediated by the activation of the right dorsolateral prefrontal cortex, which has a key role in cognitive functions in general and in the enhancement of working memory following MPH treatment in specific (Gamo et al., 2010; Marquand et al., 2012). In some embodiments, the causal relations between the observed change in the rDLPFC activation and the behavioral improvement (in terms of 3-back RT), was assessed by a mediation analysis with the attention index scores of the cognitive induction state as a mediator (Preacher & Hayes 2008). In some embodiments, a significant indirect path from rDLPFC activity to 3-back reaction-times improvement through the attention scores—an index for the performance during the cognitive induction state (indirect effect=−357.6, CI (95%)=−766.1 to −10.7) is observed, for example in FIG. 6D. In some embodiments, this effect was not found for the control conditions: placebo/cognitive, placebo/motivation, and MPH/motivation.

In some embodiments, the experimental results described in FIGS. 6A-6D suggest that successful performance of the task that activates target brain regions enhances the MPH effect via the facilitation of a stronger activation of that region. While the mechanistic characteristics of this process are yet to be detailed, this study provides a preliminary proof of concept for the benefit of FPC involving reuptake inhibitors.

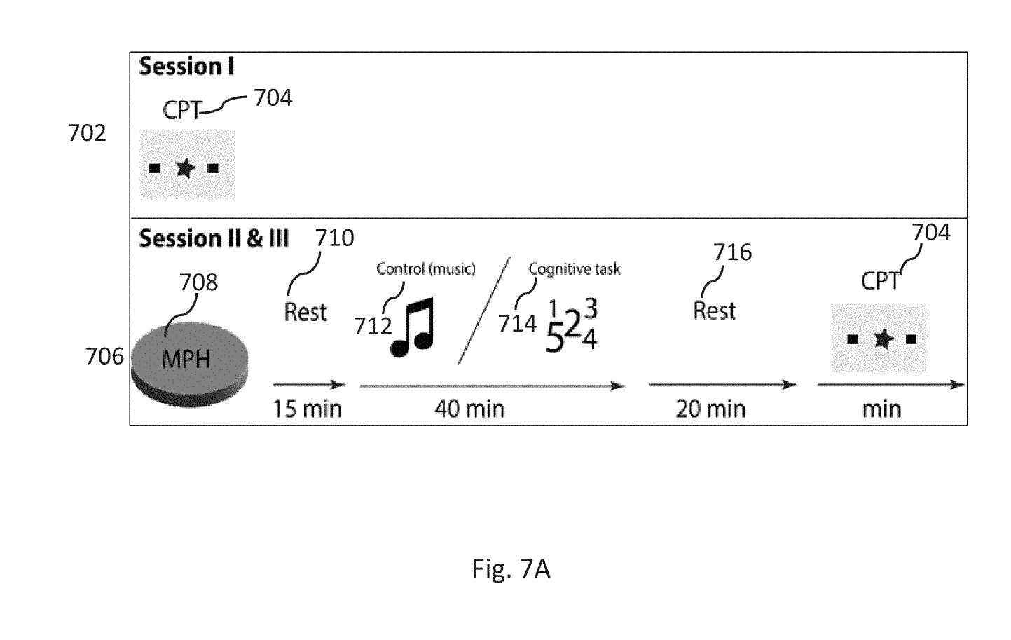

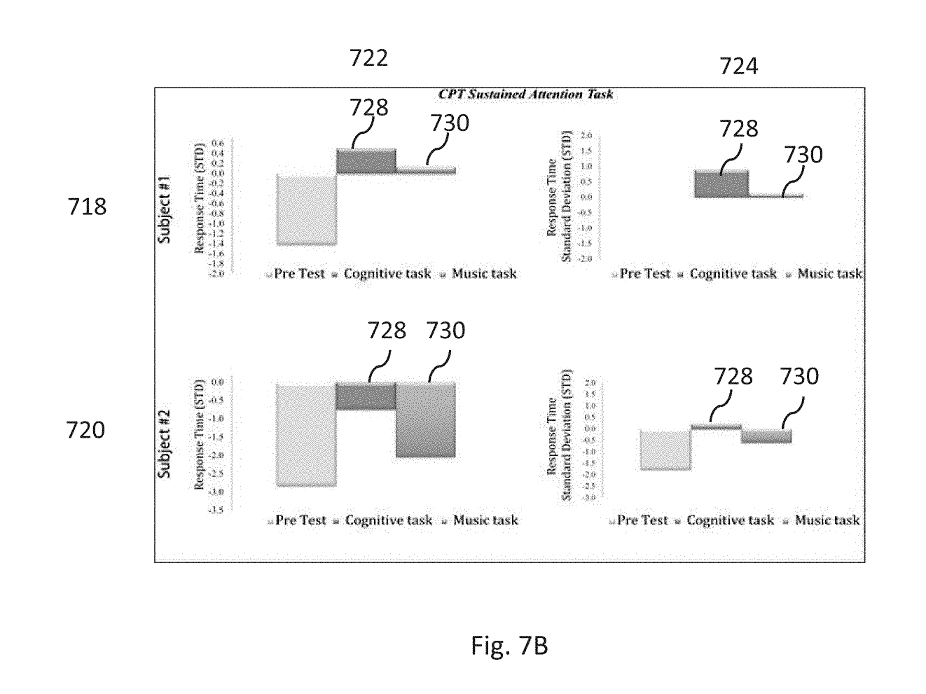

Reference is now made to FIGS. 7A-7B, depicting validation experiments for testing whether FPC improves attention functions of subjects medicated with MPH, according to some embodiments of the invention. In some embodiments, the experiment includes three sessions. In some embodiments, in the first session 702, the subjects performed a conjunctive continuous performance task (CPT, Shalev et al., 2011). In some embodiments, this test yields measures of sustained attention, which are generalized across different sensory modalities.

According to some embodiments, in the second and the third sessions 706, the subjects took half of their prescribed drug 708 dose by per os (PO) administration. In some embodiments, in the first epoch they went through either a cognitive induction condition 714 during which they performed the NeuroTrax tasks or a control condition 712 during which they listened to a piece of their favorite music. In some embodiments, these 30 min conditions were followed by a 20-minutes rest epoch 716, for example to allow for the testing of an enduring effect of the FPC. In some embodiments, an additional CPT session 704 was conducted, for example, to assess the difference between the subjects' performance after the intervention. Optionally, the order of the conditions was counterbalanced across subjects (it should be noted that CPT is not improved with experience).

According to some exemplary embodiments, FPC effect was measured for the sustained attention measures of average 722 and standard deviation 724 response time, which show high reliability and validity (Shalev et al., 2011). In some embodiments, the raw scores were converted into standard deviations computed for an independent CPT data set. In some embodiments, the sustained attention measures of average 722 and standard deviation 724 response time were improved in two subjects 718 and 720 (males; ages 28, 40) with the administration of half dose MPH. In some embodiments, the improvement was higher when the drug administration was coupled with the cognitive induction task 728 relative to the control task 730 (music).

In some embodiments, the findings presented in FIGS. 7A and 7B indicate, for example, that FPC allows for the enhancement of the advantageous pharmacological effects with reduced drug doses.

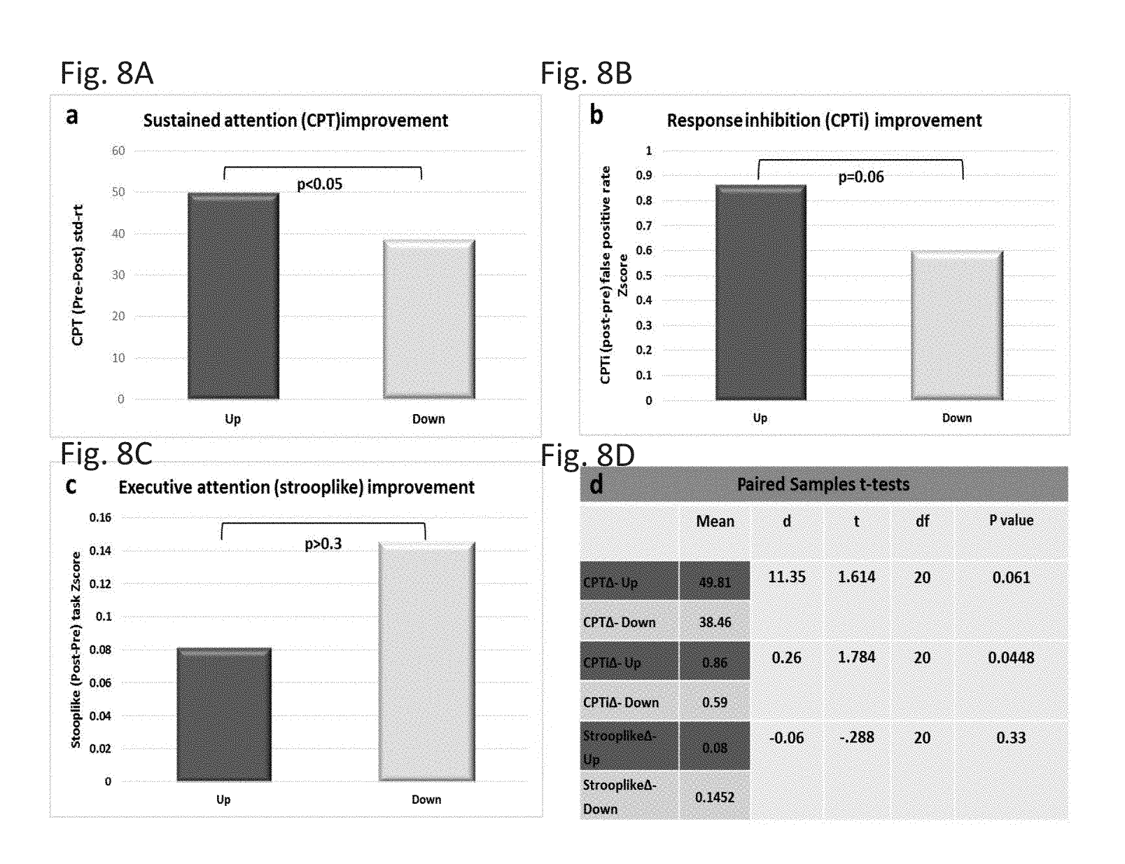

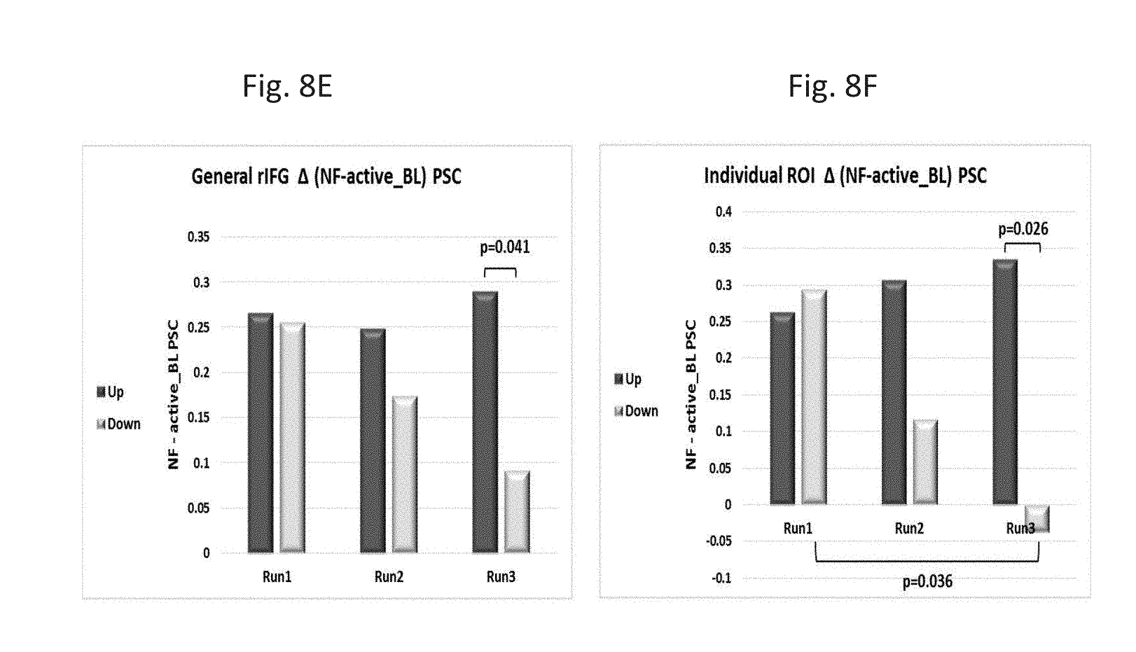

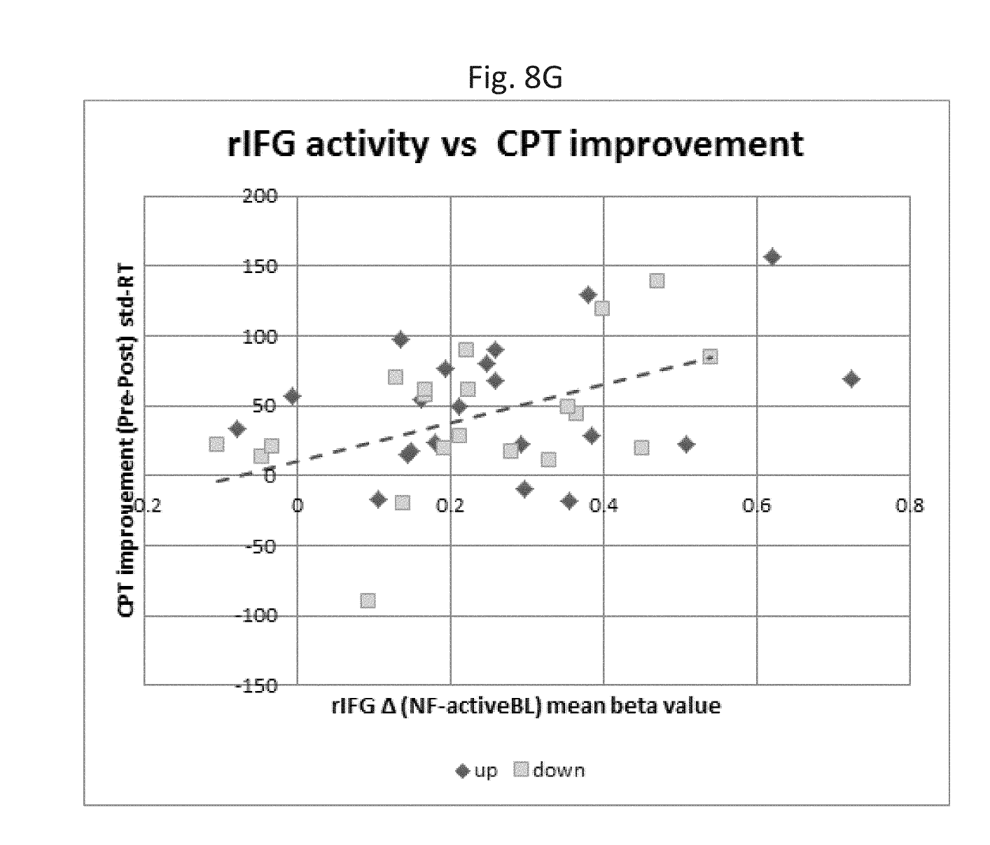

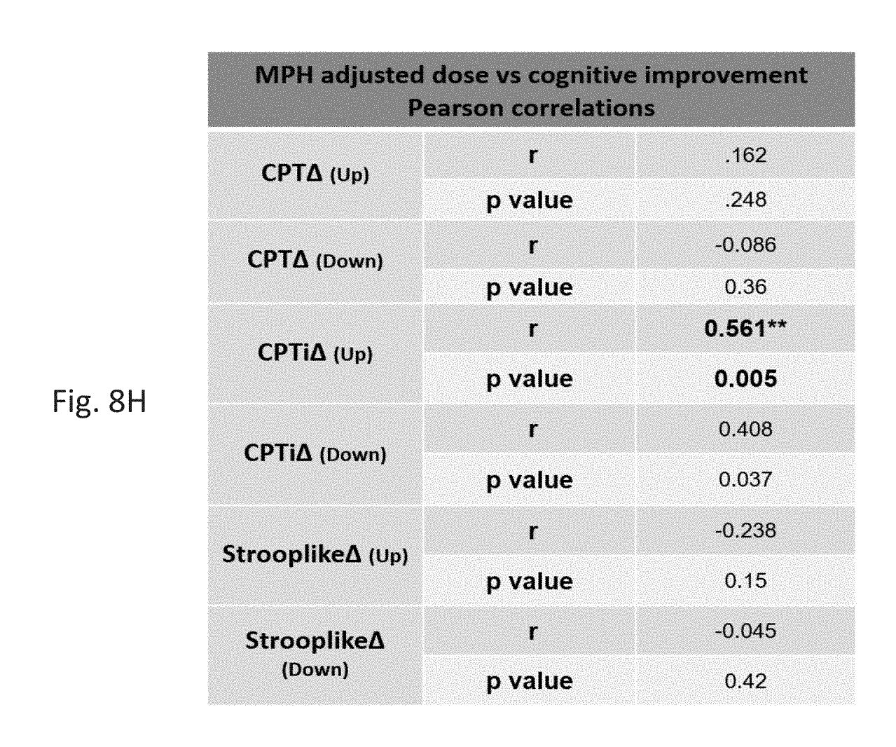

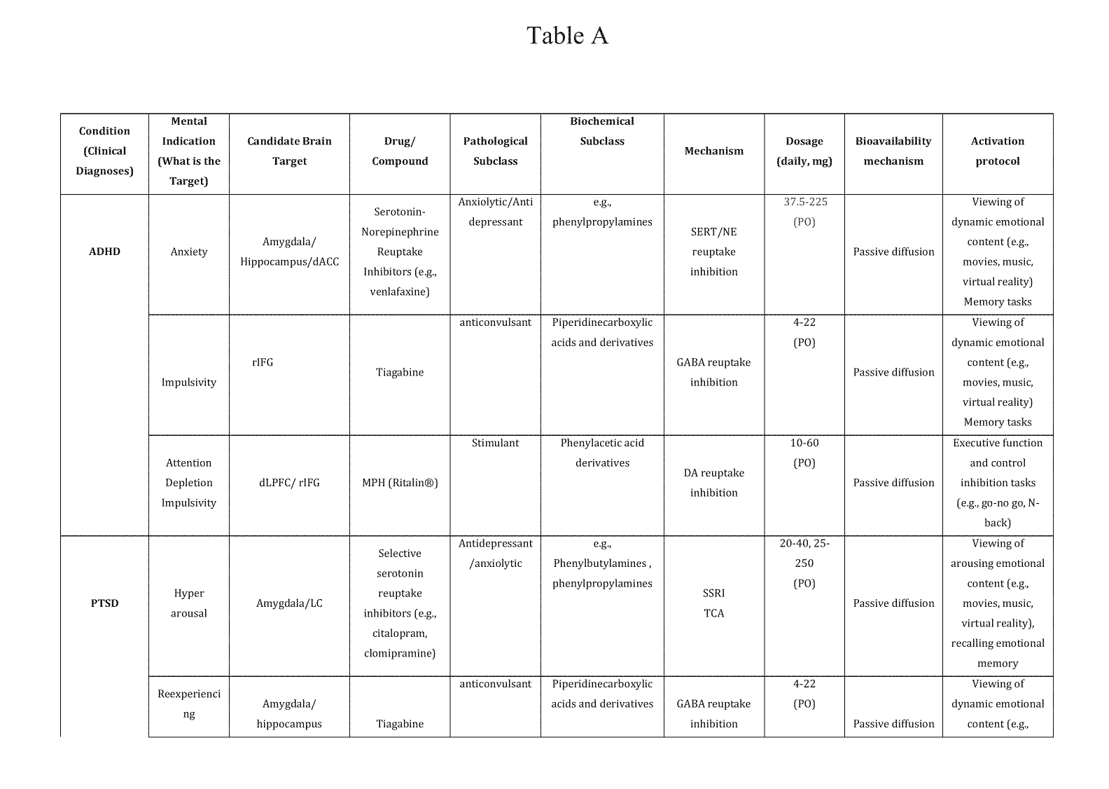

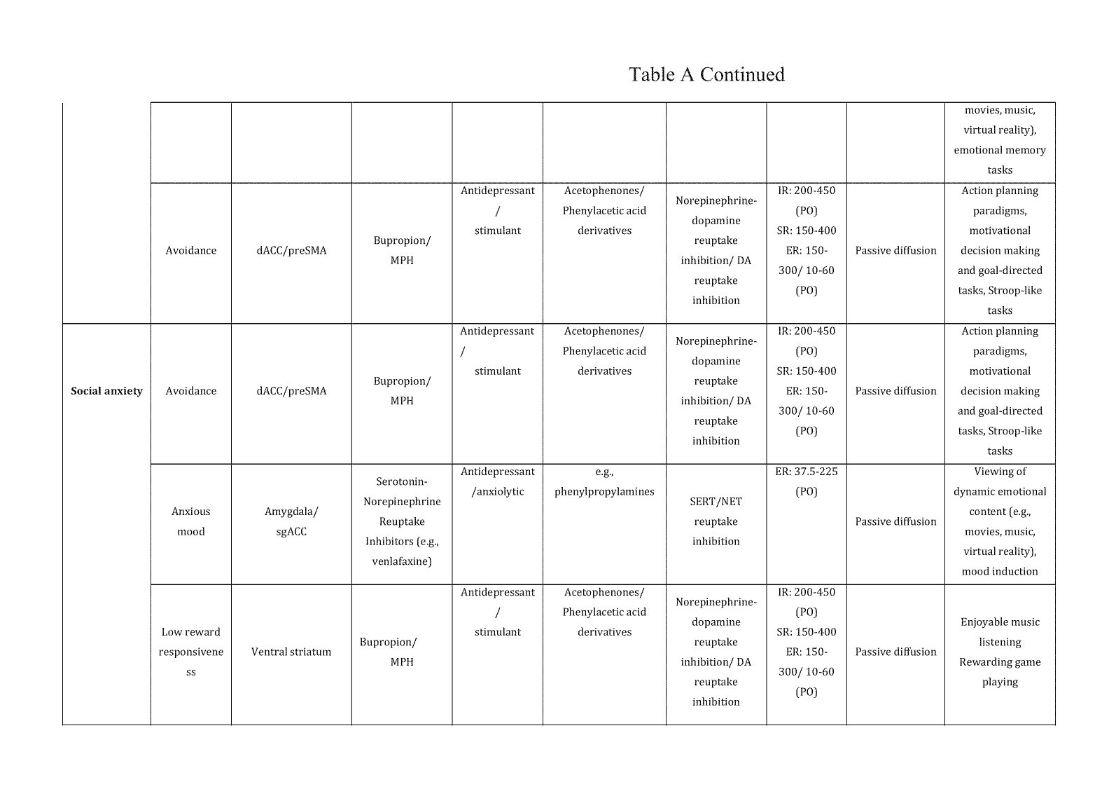

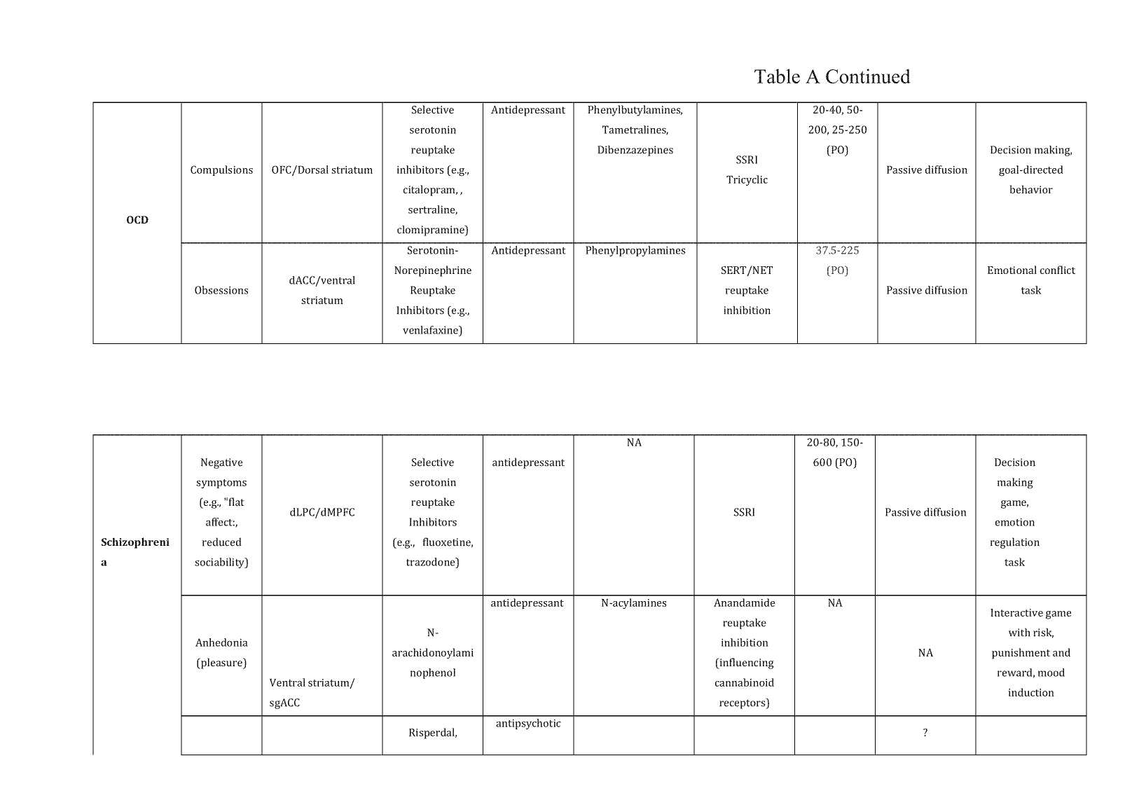

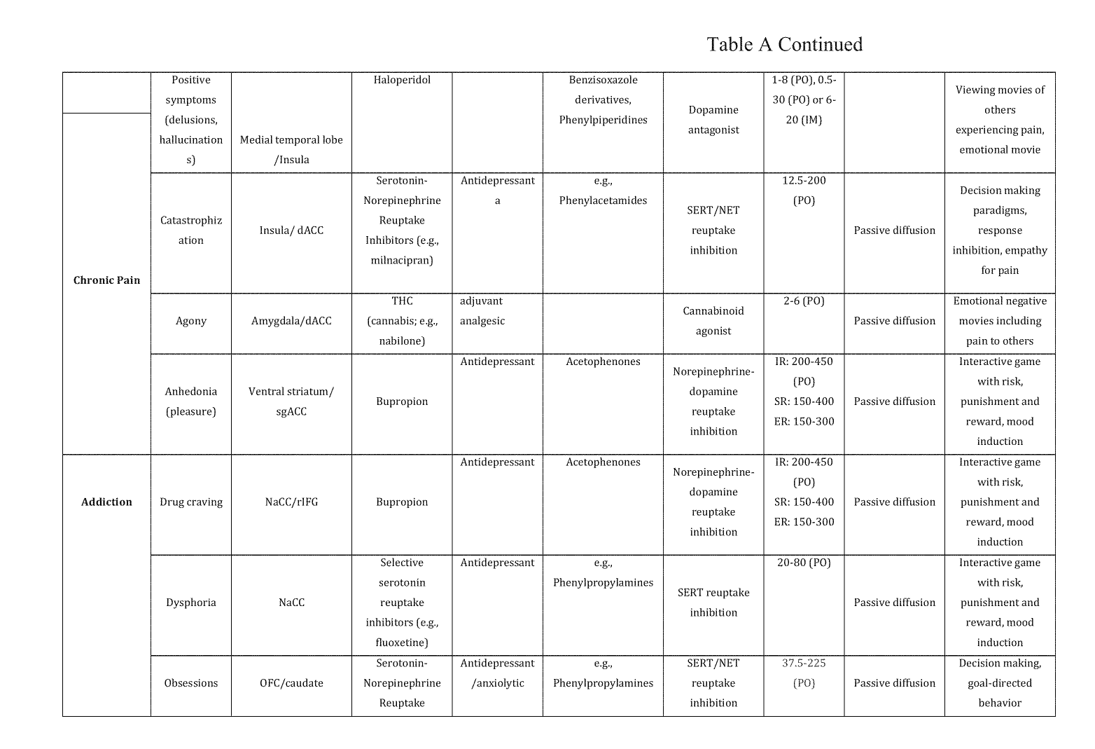

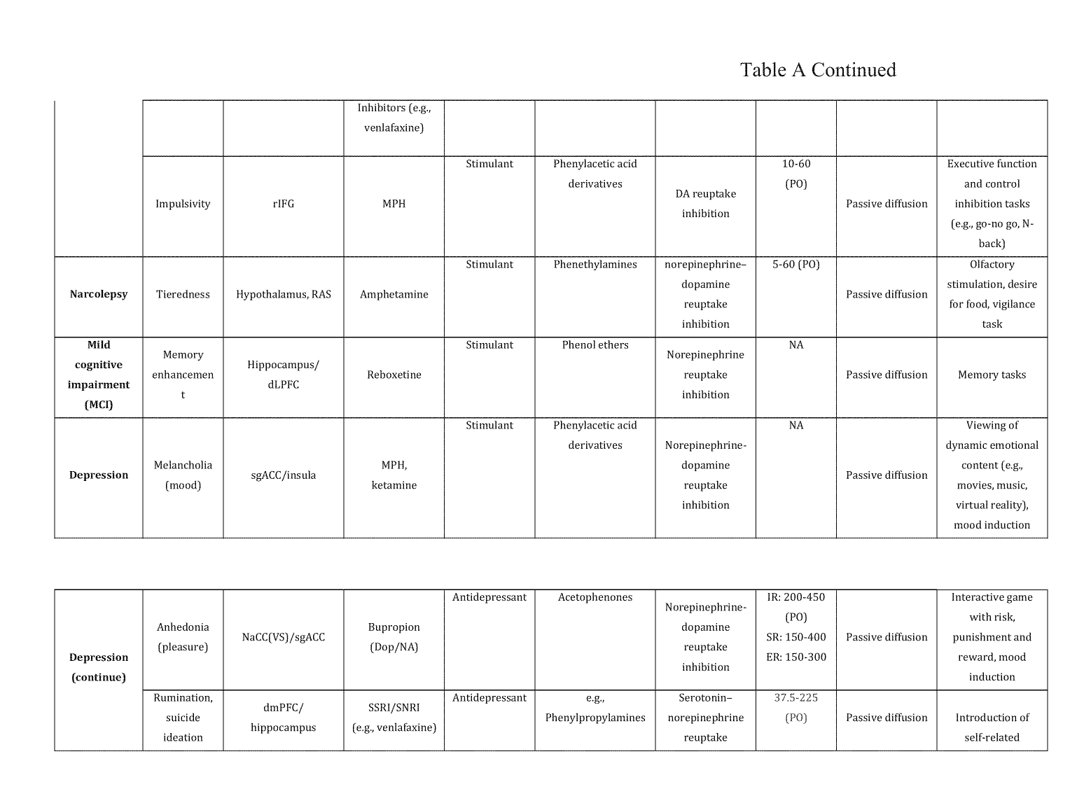

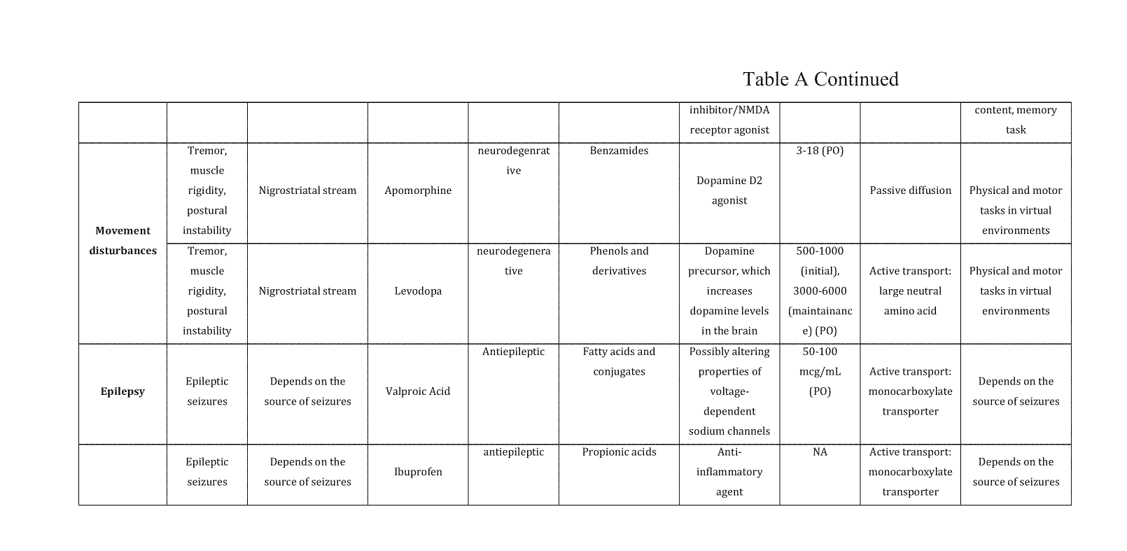

Reference is now made to FIGS. 8A-8H depicting the results of a validation experiment for measuring neurobehavioral effects of Methylphenidate and right inferior frontal gyrus neurofeedback activation combined treatment for ADHD, according to some embodiments of the invention.

Without being bound to theory, and as previously described it is plausible to assume that one could improve a psycho-pharmacological treatment by administering a drug, while inducing an advantageous physiological state in brain regions that interact with the drug's active ingredients (by selectively manipulating pharmacokinetic\dynamic properties of a drug), and that the induction of such state could be achieved with a certain kind of a behavioral task. The concept of coupling drug administration with a specific task that activates therapeutically relevant brain regions, was coined “Functional Pharmacology”.

According to some exemplary embodiments, 24 ADHD patients participated in a three session within-subject experimental design. In some embodiments, following administration of a weight-adjusted dose (0.1-0.2 mg/kg) of Ritalin IRTM (active ingredient—Methylphenidate (MPH)), they either “Up” (experimental condition) or “Down” (control condition) regulated their right-IFG (an area that mediates executive functions, and one of MPH's sites of action) BOLD activity via real-time fMRI Neurofeedback task. In some embodiments, fMRI BOLD activity is the physiological index of neuronal activity, measuring Cerebral Blood Flow and magnetic properties of the blood (Chen & Ogawa, 1996), hence, it offers an exploitable “window” to pharmacoketic manipulation.

In some embodiments, taking the known pharmacokinetic properties of MPH into account, the neurofeedback paradigm was scheduled to begin towards the estimated peak concentration of MPH in brain blood plasma (45 min), and last for 40 minutes, well into the drug's plateau of concentration phase, thus assuming a maximum effect of drug delivery and absorption to the rIFG and related brain regions. Additionally, cognitive performance was evaluated before administration and following the NF task via three behavioral tasks (CPTi, CPT and Strooplike, measuring response inhibition, sustained attention and executive attention, respectively; Shalev-Mevorach et al. 2005; 2011).

According to some embodiments, two out of three measured cognitive functions (sustained attention (CPT task) and response inhibition (CPTi task)) showed substantially larger effects following the up regulation condition, for example as shown in FIGS. 8A-8D. In some embodiments, although the manipulation of the physiological contrast created in the rIFG between up and down conditions was not absolute, but gradually increased along the sessions and peaked toward its end, for example as shown in FIGS. 8E-8F, we found a significant positive correlation between rIFG BOLD activity during the neurofeedback task and improvement in sustained attention, for example as shown in FIG. 8G. In some embodiments, response inhibition was not significantly correlated to the rIFG BOLD activity, but did exhibit a strong significant positive correlation to the drug's administered dose only in the Up regulation condition, suggesting perhaps a more complex connection between response inhibition improvement and rIFG up regulation physiological outcomes, for example as shown in FIG. 8H.

It is expected that during the life of a patent maturing from this application many relevant brain activation protocols will be developed; the scope of the term activation protocol is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for modifying the effect of a drug by application of an activation protocol, comprising:
administering a drug according to a treatment protocol;
applying an activation protocol in a timed relationship to said administering, for differentially activation of at least one selected brain region;
wherein said differentially activation allows said drug to selectively interact with said at least one selected brain region.

2. (canceled)

3. The method of claim 1, wherein said applying further comprises applying an activation protocol before and/or after said administering.

4. (canceled)

5. The method of claim 1, further comprising determining a brain activation profile prior to and/or following said applying.

6. The method of claim 5, further comprising modifying said activation protocol according to said determining.

7. The method of claim 6 wherein said modifying comprises modifying said activation protocol to reach desired activation levels of at least one specific region and/or desired connectivity measures of at least one neural network by explicit or covert neurofeedback.

8. The method of claim 5, further comprising modifying said drug dosage according to said determining.

9. The method of claim 1, further comprising determining the effect of said drug by measuring at least one clinical parameter value, following said applying.

10. (canceled)

11. The method of claim 9, further comprising modifying said activation protocol if said effect of said drug is not a desired effect.

12. (canceled)

13. The method of claim 1, wherein said timed relationship is adjusted according to said effect and/or according to said drug.

14. (canceled)

15. The method of claim 9, wherein said effect comprises reducing at least one side effect of said drug.

16. The method of claim 1, wherein said drug is methylphenidate and wherein said treatment protocol is used to treat ADHD.

17-19. (canceled)

20. The method of claim 1, wherein said drug is Levodopa and wherein said treatment protocol is used to treat PD.

21-23. (canceled)

24. The method of claim 1, wherein said drug is Haloperidol and wherein said treatment protocol is used to treat Schizophrenia.

25-27. (canceled)

28. The method of claim 1, wherein said applying an activation protocol comprises performing a neurofeedback protocol.

29. (canceled)

30. The method claim 1, wherein said applying an activation protocol comprises performing at least one executive function task and/or at least one control inhibition task and/or at least one action planning paradigm.

31-32. (canceled)

33. A method for treating a neurological disease, comprising:
administering a drug according to a treatment protocol;
applying an activation protocol in a timed relationship to said administering, for differentially activation of at least one selected brain region;
wherein said differentially activation allows said drug to selectively interact with said at least one selected brain region.

34. (canceled)

35. The method according to claim 33, wherein said applying comprises applying said activation protocol prior to and/or after said administering.

36. The method of claim 33, further comprising determining a brain activation profile prior to said applying.

37. (canceled)

38. The method of claim 36, further comprising modifying said activation protocol according to said determining.

39. The method of claim 36, further comprising modifying said drug dosage according to said determining.

40-80. (canceled)
